(19) United States
(12) Patent Application Publication
(10) Pub. No.: US 2026/0026792 A1
(43) Pub. Date: Jan. 29, 2026

(54) DEVICE ALLOWING LARGE BORE TRANSSEPTAL ACCESS WITH SUBSEQUENT ATRIAL RE-ACCESS AND METHOD THEREOF

(71) Applicant: AMX Technologies, LLC, Plymouth, MN (US)

(72) Inventors: Jaime E. SARABIA, Plymouth, MN (US); Richard FARRELL, Plymouth, MN (US); Daniel P. COYLE, Plymouth, MN (US)

(73) Assignee: AMX Technologies, LLC, Plymouth, MN (US)

(21) Appl. No.: 19/349,872

(22) Filed: Oct. 3, 2025

Related U.S. Application Data
(63) Continuation of application No. 18/000,103, now abandoned.
(60) Provisional application No. 63/036,435, filed on Jun. 8, 2020.

(51) Int. Cl. A61B 17/00; A61B 17/04; A61B 17/22

(52) U.S. Cl. CPC A61B 17/0057; A61B 17/0401; A61B 2017/00623; A61B 2017/00663; A61B 2017/22044

(57) ABSTRACT

The present disclosure relates to medical devices. More particularly, this disclosure describes a vascular device allowing large bore transseptal access with subsequent atrial re-access by preplacing closures/tissue approximating sutures prior to creating a septostomy. Generally, the device may include a delivery catheter for puncturing and cutting the interatrial septum. An anchor of the delivery catheter may secure the suture in an atrium to a septum wall, for example, the left atrium. Incisions may be made by an expandable cutting implement which may use mechanical or radio frequency (RF) energy without interfering with the suture. A therapeutic instrument may be advanced through the tissue plane after the incisions are made by the cutting implement. Closure of the incision may be performed with the previously placed sutures.

### RELATED APPLICATIONS

This application is the continuation of and claims priority to Ser. No. 18/000,103 filed Nov. 28, 2022, entitled Device Allowing Large Bore Transseptal Access With Subsequent Atrial Re-Access And Method Thereof, which is the U.S. National Phase of and claims priority to International Patent Application No. PCT/US2020/041853, International Filing Date Jul. 13, 2020, entitled Device Allowing Large Bore Transseptal Access With Subsequent Atrial Re-Access And Method Thereof; which claims the benefit of and priority to U.S. Provisional Application Ser. No. 63/036,435 filed Jun. 8, 2020 entitled Large Bore Septal Closure; all of which are incorporated herein by reference in their entireties.

### TECHNICAL FIELD

The present disclosure relates to medical devices and more particularly, to transcatheter-delivered interatrial septal crossing and closing techniques for a large bore instrument allowing access into an atrium (e.g., the left atrium) with subsequent atrial re-access.

### BACKGROUND

An increasingly common approach for left heart catheter procedures may be to puncture and cross an interatrial septum using a mechanical or radio frequency (RF) powered needle. This procedure is generally straightforward for small bore catheters, which are typically less than 24 French. If larger catheters or bores, however, are to be sent across the atrial septum, a puncture site dilation is typically used in order to advance the catheter through the septum. Current methodologies for dilating the initial septal puncture site may involve the use of a dilator, or by inflating a balloon, to open the access site. This may use multiple tool exchanges by the physician and may have undesirable consequences on the tissue due to the uncontrolled nature of the dilation techniques.

In addition, minimally-invasive, catheter-based therapies are being developed that allow physicians to provide treatments to patients whose existing comorbidities may preclude them from having a needed, but more invasive, surgical procedure. Over the last few years, catheter based procedures have developed which may involve implantation of repair or replacement mitral valves, which may use large bore transseptal access. The transseptal puncture may result in the formation of an iatrogenic atrial septal defect which may need to be subsequently closed by an atrial septal defect device. However, that atrial septal defect device may preclude, or make difficult, subsequent transseptal crossing.

The present disclosure provides for a device allowing large bore transseptal access with subsequent atrial re-access and method thereof that addresses the above identified concerns. A controlled and precise atrial septostomy that permits passage of the large bore device across the interatrial septum and then provides a rapid and permissive closure of the procedurally created atrial septal defect is described herein. The word permissive may be defined as a mechanism which the septal defect is closed and may allow future crossings of the interatrial septum by standard transseptal methods. Other benefits and advantages will become clear from the disclosure provided herein and those advantages provided are for illustration. The statements in this section merely provide the background related to the present disclosure and does not constitute prior art.

### SUMMARY

This summary is provided to introduce a selection of concepts in a simplified form that are further described below in the DESCRIPTION OF THE DISCLOSURE. This summary is not intended to identify key features of the claimed subject matter, nor is it intended to be used as an aid in determining the scope of the claimed subject matter.

According to one aspect of the present disclosure, a vascular device for performing a transseptal puncture is provided. The device may include a body, an anchor extending from a distal end of the body through a shaft disposed within the body, at least one suture coupled to at least one needle within the anchor, at least one catch extending from the body to pull the at least one needle into the body for placing the at least one suture, and a cutting implement between the body and anchor coupled to an actuating shaft aligned with the at least one suture.

According to another aspect of the present disclosure, a septal orifice closure apparatus allowing re-access is provided. The apparatus may include a body on a first side of a septal orifice in a septum of a heart, an anchor on a second side of the septal orifice extending from a distal end of the body through a shaft disposed within the body, at least one suture coupled to at least one needle disposed within the anchor, at least one catch extending from the body to pull the at least one needle into the body for placing the at least one suture, and a cutting implement between the body and anchor coupled to an actuating shaft aligned with the at least one suture.

According to yet another aspect of the present disclosure, a vascular closure apparatus is provided. The apparatus may include an anchor positioned through a puncture in a vessel wall and operable between retracted and expanded positions from a body, at least one suture disposed within the anchor, at least one needle coupled to the at least one suture extending through the vessel wall adjacent to the puncture to connect the at least one suture when the anchor is in the expanded position, at least one catch extending from the body to pull the at least one needle into the body for placing the at least one suture, and a cutting implement between the body and anchor coupled to an actuating shaft aligned with the at least one suture.

According to another aspect of the present disclosure, a method of performing a septal crossing in a vessel wall is provided. The method may include providing a delivery catheter having a body and an anchor, inserting the anchor through a puncture in the vessel wall, operating the anchor into an expanded position capturing the vessel wall between the body and the anchor to expose at least one needle, capturing the at least one needle through the vessel wall adjacent to the puncture and into engagement with at least one suture, and positioning the at least one suture in the vessel wall.

According to one aspect of the present disclosure, a vascular apparatus is provided. The apparatus may include a delivery system having at least one anchor penetrating a tissue plane, the at least one anchor having a suture, a cutting implement positioned into the tissue plane facilitating an incision, a therapeutic instrument advanced into the incision, and a fastener securing the suture with tissue of the tissue plane.

According to yet another aspect of the present disclosure, a septal orifice closure apparatus is provided. The apparatus may include a first pledget introduced into a tissue plane through a cannula, wherein the first pledget is coupled to a control line tensioning the first pledget after introduction into the tissue plane, a second pledget introduced into the tissue plane through the cannula, wherein the second pledget is coupled to a control line tensioning the second pledget after introduction into the tissue plane, a cutting implement making an incision between the first pledget and second pledget, a therapy device passed through the incision, and a knot made of the control line of the first pledget and the control line of second pledget tensioning the first pledget and second pledget with tissue from the tissue plane therebetween.

According to another aspect of the present disclosure, a device for puncturing an atrial septum of a patient is provided. The device may include a body, a tip extending from a distal end of the body, and a cutting member in a collapsed state disposed between the body and tip, wherein the tip followed by the cutting member penetrates into a tissue plane, the cutting member expanded after passing through the tissue plane.

According to one aspect of the present disclosure, a vascular apparatus is provided. The apparatus may include a delivery system, a tip extending from a distal end of the delivery system, and a cutting implement disposed between the delivery system and tip.

According to another aspect of the present disclosure, a method of instrumenting the left atrium is provided. The method may include puncturing a septum with a needle, placing at least one suture behind the septum, advancing a therapeutic instrument into the puncture, and cinching the at least one suture closing the puncture.

According to another aspect of the present disclosure, a method of closing a septal orifice is provided. The method may include creating a transseptal access through a wire, inserting a delivery catheter over the wire, enlarging the transseptal access through a cutting implement of the delivery catheter, inserting at least one suture coupled to a needle that passes around the transseptal access, cinching the transseptal access with the at least one suture, and removing the delivery catheter.

### BRIEF DESCRIPTION OF THE DRAWINGS

The novel features believed to be characteristic of the disclosure are set forth in the appended claims. In the descriptions that follow, like parts are marked throughout the specification and drawings with the same numerals, respectively. The drawing FIGURES are not necessarily drawn to scale and certain FIGURES may be shown in exaggerated or generalized form in the interest of clarity and conciseness. The disclosure itself, however, as well as a preferred mode of use, further objectives and advantages thereof, will be best understood by reference to the following detailed description of illustrative embodiments when read in conjunction with the accompanying drawings, wherein:

FIG. 1 is a front view schematic representation of an illustrative human venous circulatory system of a patient with a guidewire routed from the femoral vein into the right atrium in accordance with one aspect of the present disclosure;

FIG. 2 is a front view schematic representation of the illustrative human venous circulatory system of the patient with an exemplary vascular device advanced into a right atrium in accordance with one aspect of the present disclosure;

FIG. 3 is a cross-sectional illustration of a heart with the exemplary vascular device positioned at an atrial septum and a septal penetrator advanced across the atrial septum into a left atrium in accordance with one aspect of the present disclosure;

FIG. 4 is a cross-sectional illustration of the heart with the exemplary vascular device advanced into the left atrium across the atrial septum and the septal penetrator withdrawn in accordance with one aspect of the present disclosure;

FIG. 5 is a cross-sectional illustration of the heart showing illustrative initial incisions followed by lengthening them in accordance with one aspect of the present disclosure;

FIG. 6 is an illustrative side view of the exemplary vascular device in accordance with one aspect of the present disclosure;

FIG. 7 is an isometric view of a distal end of the exemplary vascular device in a low profile condition in accordance with one aspect of the present disclosure;

FIG. 8 is an isometric view of the distal end of the exemplary vascular device viewed from a different perspective with a portion of the device advanced revealing components used to puncture tissue and pass sutures in accordance with one aspect of the present disclosure;

FIG. 9 is an isometric view of the distal end of the exemplary vascular device with the portion of the device advanced through the tissue of the atrial septum in accordance with one aspect of the present disclosure;

FIG. 10 is a cross sectional view of the exemplary vascular device having an illustrative lumen configuration in accordance with one aspect of the present disclosure;

FIG. 11 is an isometric, sectioned view of the distal end of the exemplary vascular device revealing the inner geometry of components therein in accordance with one aspect of the present disclosure;

FIG. 12 is an isometric view of the distal end of the exemplary vascular device revealing snares that have been advanced out of a body of the vascular device in accordance with one aspect of the present disclosure;

FIG. 13 is an isometric view of the distal end of the exemplary vascular device after four suture needles and suture ends have been passed through the tissue, snared and pulled through the length of the device in accordance with one aspect of the present disclosure;

FIG. 14 is an isometric view of the distal end of the exemplary vascular device after the suture has been pulled completely through the length of the device and is now pulled taught against the tissue in accordance with one aspect of the present disclosure;

FIG. 15 is an isometric view of the distal end of the vascular device after a cutting implement has been partially advanced revealing the cutting elements in accordance with one aspect of the present disclosure;

FIG. 16 is an isometric view of the distal end of the vascular device with the cutting implement expanded in accordance with one aspect of the present disclosure;

FIG. 17. is an isometric view of the exemplary vascular device positioned to cut the tissue in accordance with one aspect of the present disclosure;

FIG. 18 is an isometric view of sutures that would be inserted into the tissue in accordance with one aspect of the present disclosure;

FIG. 19 is an isometric view of an incision after the tissue is engaged with the suture in accordance with one aspect of the present disclosure;

FIGS. 20A-L are various illustrative cut patterns that may be created using the vascular device in the atrial septum which may be made from at least one cutting implement that may be rotated and used multiple times in accordance with one aspect of the present disclosure;

FIG. 21 is an isometric view of an illustrative cut pattern for incision to promote tissue edge apposition in accordance with one aspect of the present disclosure;

FIG. 22 is an isometric view of the illustrative cut pattern for incision to promote the tissue edge apposition, while under slight tension, demonstrating tissue edge control and overlap of edges with tension applied in accordance with one aspect of the present disclosure;

FIG. 23 is an isometric view of the cut patterns for incision to promote the tissue edge apposition and an illustrative helical anchor controlling tissue edges in accordance with one aspect of the present disclosure;

FIG. 24 is an isometric view of an illustrative expandable radio frequency (RF) cutting implement with four expandable members in accordance with one aspect of the present disclosure;

FIG. 25 is an isometric view of an exemplary vascular device with the expandable cutting implement having a tip for piercing the tissue in accordance with one aspect of the present disclosure;

FIG. 26 is an isometric view of the exemplary vascular device with the illustrative expandable cutting implement placed beyond the tissue in accordance with one aspect of the present disclosure;

FIG. 27 is an isometric view of the exemplary vascular device with the illustrative expandable cutting implement making incisions into the tissue in accordance with one aspect of the present disclosure;

FIG. 28 is an isometric view of the exemplary vascular device with the illustrative expandable cutting implement advancing anchor mechanisms in accordance with one aspect of the present disclosure;

FIG. 29 is an isometric view of the exemplary vascular device with the illustrative expandable cutting implement with push anchors further inserted to advance the anchor mechanisms in accordance with one aspect of the present disclosure;

FIG. 30 is an isometric view of the exemplary vascular device having the anchor mechanisms removed in accordance with one aspect of the present disclosure;

FIG. 31 is an isometric view of the exemplary cutting implement removed from the tissue leaving tissue anchors against it in accordance with one aspect of the present disclosure;

FIG. 32 is an isometric view of an exemplary toggle within the tissue in accordance with one aspect of the present disclosure;

FIG. 33 is an isometric view of an illustrative cutting implement having an atraumatic tip deployed from an exemplary vascular device in accordance with one aspect of the present disclosure;

FIG. 34 is an isometric view of an illustrative cutting implement having a slit in a sheath deployed from an exemplary vascular device in accordance with one aspect of the present disclosure;

FIG. 35 is a side view of the illustrative cutting implement having a cutting element extending from the slit in the sheath in accordance with one aspect of the present disclosure;

FIG. 36 is an isometric view of a distal end of the exemplary cutting implement in accordance with one aspect of the present disclosure;

FIG. 37 is a cross-sectional illustration of the cutting implement in accordance with one aspect of the present disclosure;

FIGS. 38A-E are schematics showing how the exemplary cutting implements may be used to optimize cutting performance and minimize power input in accordance with one aspect of the present disclosure;

FIG. 39 is an isometric view of exemplary tissue being crossed using an illustrative guidewire in accordance with one aspect of the present disclosure;

FIG. 40 is an isometric view of suture anchors in delivery sheaths in accordance with one aspect of the present disclosure;

FIG. 41 is an isometric view of illustrative suture anchors in delivery sheaths about to penetrate the tissue in accordance with one aspect of the present disclosure;

FIG. 42 is an isometric view of the illustrative suture anchors in delivery sheaths engaging or penetrating the tissue in accordance with one aspect of the present disclosure;

FIG. 43 is an isometric view of the illustrative suture anchors in delivery sheaths engaging the tissue with suture control lines attached in accordance with one aspect of the present disclosure;

FIG. 44 is an isometric view of an illustrative cutting implement in a sheathed position in accordance with one aspect of the present disclosure;

FIG. 45 is an isometric view of the illustrative cutting implement in an unsheathed position in accordance with one aspect of the present disclosure;

FIG. 46 is an isometric view of the illustrative cutting implement in an unsheathed position and expanded in accordance with one aspect of the present disclosure;

FIG. 47 is an isometric view of the illustrative cutting implement making an incision or cut into the tissue in accordance with one aspect of the present disclosure;

FIG. 48 is an isometric view of an illustrative incision or cut in the tissue with a guidewire passing through it in accordance with one aspect of the present disclosure;

FIG. 49 is an isometric view of an advancing of an exemplary therapeutic instrument passing through the cut in the tissue over the guidewire in accordance with one aspect of the present disclosure;

FIG. 50 is an isometric view of an illustrative tissue anchor lock with helical barbs in accordance with one aspect of the present disclosure;

FIG. 51 is an isometric view of the illustrative tissue anchor lock with helical barbs engaged in the tissue passed over the suture control lines in accordance with one aspect of the present disclosure;

FIG. 52 is an isometric view of the illustrative tissue anchor lock with helical barbs engaged in the tissue passed over the suture control lines and the control lines trimmed to the level of the anchor in accordance with one aspect of the present disclosure;

FIG. 53 is an isometric view of the illustrative tissue anchor lock with helical barbs engaged in an other side of the tissue in accordance with one aspect of the present disclosure;

FIG. 54 is an isometric view of an illustrative pledget made from biocompatible or bio-absorbable material in accordance with one aspect of the present disclosure;

FIG. 55 is an isometric view of an exemplary cannula piecing heart tissue in accordance with one aspect of the present disclosure;

FIG. 56 is an isometric view of the exemplary cannula piecing heart tissue and the illustrative pledget made from biocompatible or bioabsorbable material advanced through the cannula in accordance with one aspect of the present disclosure;

FIG. 57 is an isometric view of the exemplary cannula piecing heart tissue and the illustrative pledget made from biocompatible or bioabsorbable material advanced out of the cannula in accordance with one aspect of the present disclosure;

FIG. 58 is an isometric view of the exemplary cannula piecing heart tissue and the illustrative pledget made from biocompatible or bioabsorbable material advanced out of the cannula and a control line tensioned to shorten the pledget in accordance with one aspect of the present disclosure;

FIG. 59 is an isometric view of the illustrative pledget made from biocompatible or bioabsorbable material tensioned to shorten the pledget with the exemplary cannula piecing heart tissue withdrawn and retained on a heart tissue surface in accordance with one aspect of the present disclosure;

FIG. 60 is an isometric view of an illustrative concentric pledget made from biocompatible or bioabsorbable material in accordance with one aspect of the present disclosure;

FIG. 61 is an isometric view of the exemplary cannula piecing heart tissue next to the pledget in accordance with one aspect of the present disclosure;

FIG. 62 is an isometric view of the exemplary cannula piecing heart tissue and the illustrative concentric pledget made from biocompatible or bioabsorbable material advanced out of the cannula in accordance with one aspect of the present disclosure;

FIG. 63 is an isometric view of the exemplary cannula piecing heart tissue and the illustrative concentric pledget made from biocompatible, or bioabsorbable material advanced out of the cannula and a control tensioned to shorten the pledget in accordance with one aspect of the present disclosure;

FIG. 64 is an isometric view of the illustrative concentric pledget made from biocompatible or bioabsorbable material tensioned to shorten the pledget and an exemplary incision made between the pledgets in accordance with one aspect of the present disclosure;

FIG. 65 is an isometric view of an exemplary therapeutic instrument placed into the incision between the pledgets into the tissue in accordance with one aspect of the present disclosure;

FIG. 66 is an isometric view of an illustrative knot advanced up to a heart tissue with the two pledget control lines in accordance with one aspect of the present disclosure;

FIG. 67 is an isometric view of the illustrative knot advanced up to the heart tissue with the two pledget control lines and tightened from a knot side of the tissue in accordance with one aspect of the present disclosure;

FIG. 68 is an isometric view of the illustrative knot advanced up to the tissue with the two pledget control lines tightened from the pledget side in accordance with one aspect of the present disclosure;

FIG. 69 is an isometric view of the illustrative incision closed between the pledgets in accordance with one aspect of the present disclosure; and

FIG. 70 is an illustrative flow chart showing exemplary processes for allowing a large bore transseptal access with subsequent atrial re-access in accordance with one aspect of the present disclosure.

### DESCRIPTION OF THE DISCLOSURE

The description set forth below in connection with the appended drawings is intended as a description of exemplary embodiments of the disclosure and is not intended to represent the only forms in which the present disclosure may be constructed and/or utilized. The description sets forth the functions and the sequence of blocks for constructing and operating the disclosure in connection with the illustrated embodiments. It is to be understood, however, that the same or equivalent functions and sequences may be accomplished by different embodiments that are also intended to be encompassed within the spirit and scope of this disclosure.

The present disclosure relates to medical devices. More particularly, this disclosure describes a vascular device allowing large bore transseptal access with subsequent atrial re-access by preplacing closures/tissue approximating sutures prior to creating a septostomy. Generally, the device may include a delivery catheter for puncturing and cutting the interatrial septum. An anchor of the delivery catheter may secure the suture in an atrium to a septum wall, for example, the left atrium. Incisions may be made by an expandable cutting implement which may use mechanical or radio frequency (RF) energy without interfering with the suture. The suture may be made of a high temperature resistant material to prevent damage if it is in contact with the cutting implement. A therapeutic instrument may be advanced through the tissue plane after the incisions are made by the cutting implement. Closure of the incision may be performed with the previously placed sutures.

Numerous other modifications or configurations to the vascular device will become apparent from the description provided below. For example, closing the incision of the interatrial septum may involve needles that may puncture the septum and pass push anchors into the tissue. Control lines tied to two or more pledgets may be also used and placed through the tissue plane via a cannula to promote tissue edge overlap and apposition.

Advantageously, the initial puncture with suture management nearby allows for a rapid closure of the procedurally created iatrogenic atrial septal defect (ASD) while permissively allowing multiple instruments within the single delivery catheter access at the atrial septum. Incisions made therefrom are easily cinched allowing re-access through the anchors/sutures. The vascular device may be useful in procedures requiring large bore trans-venous access to a left atrium for transcatheter mitral valve replacement, where the delivery systems commonly create a large residual ASD. Other benefits and advantages will become clear from the disclosure provided herein and those advantages provided are for illustration.

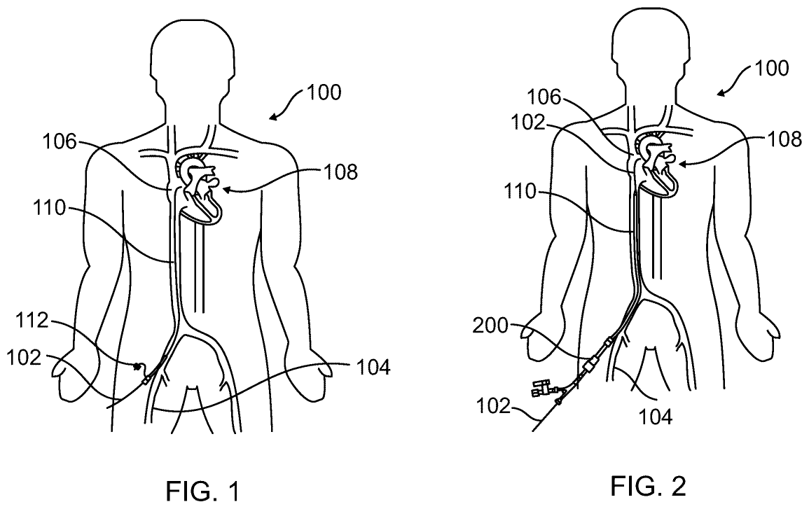

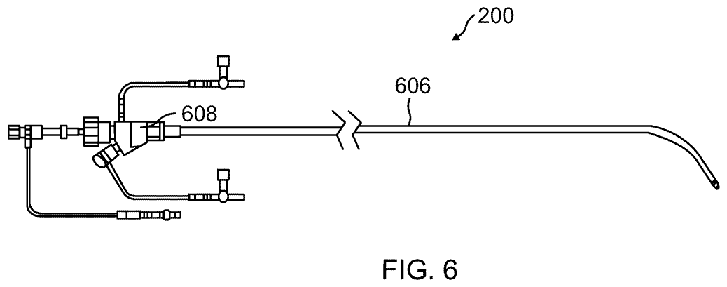

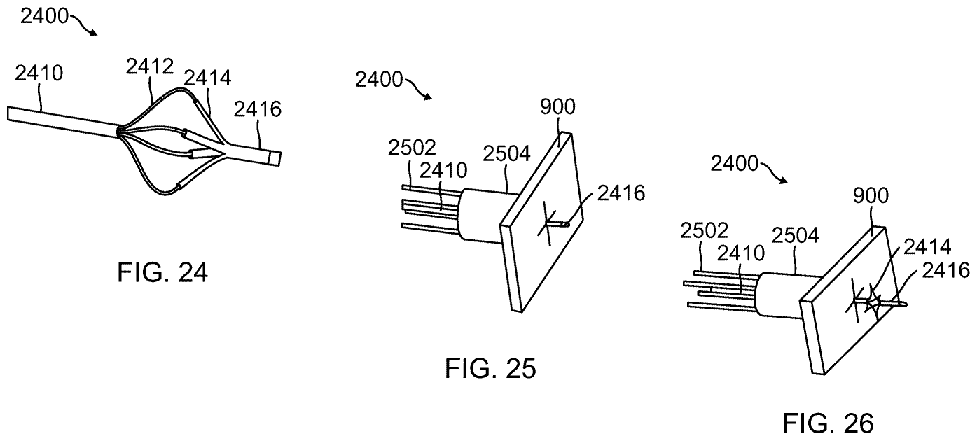

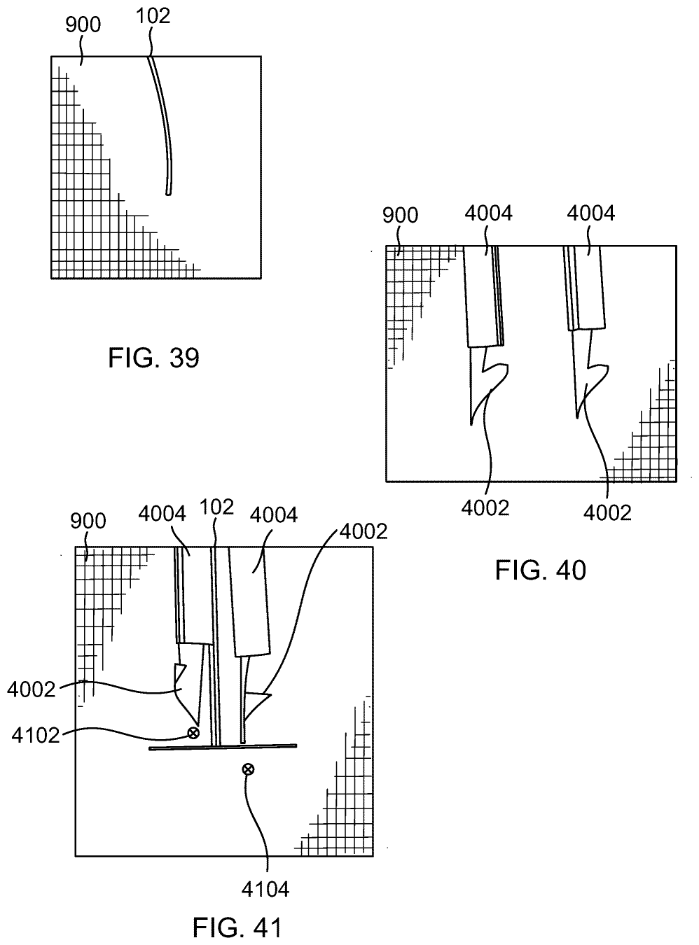

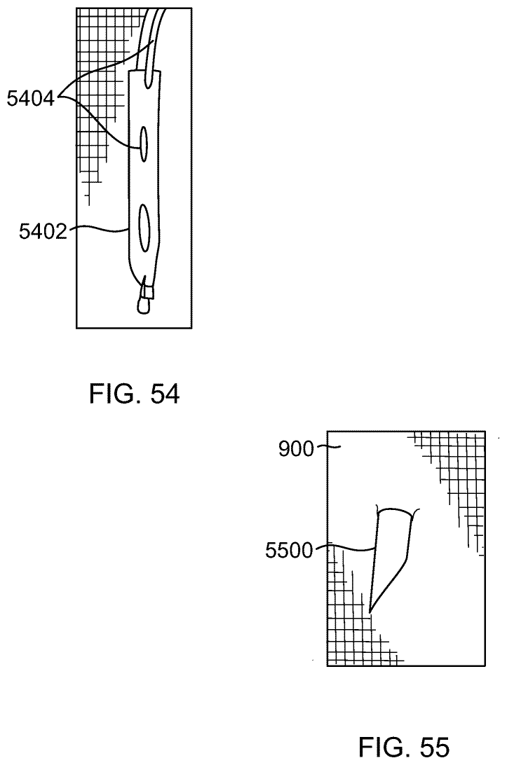

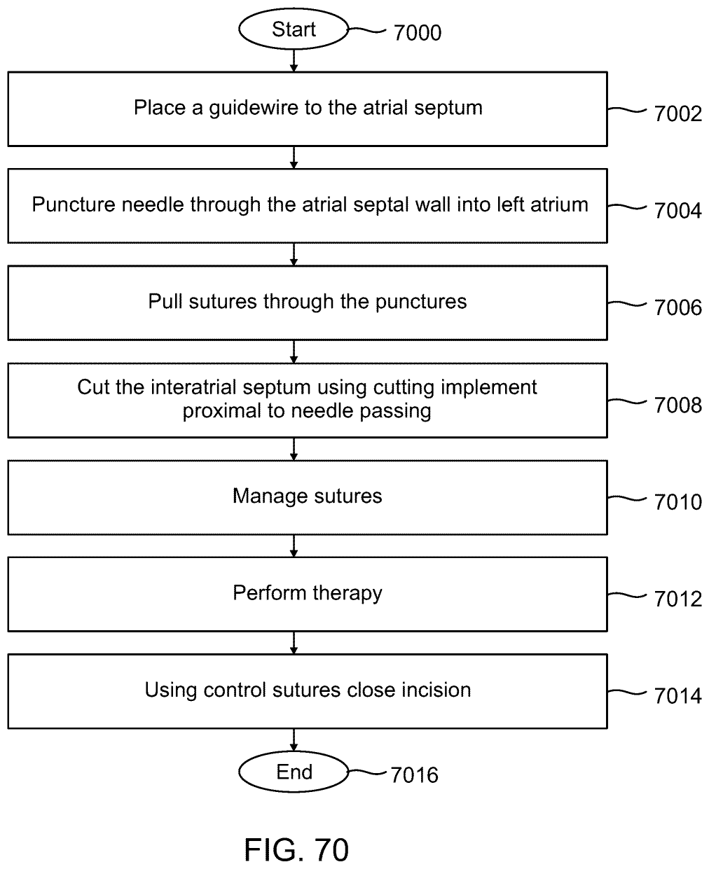

FIGS. 1-5 represent a human venous circulatory system including a heart with an exemplary vascular device with guidewire defined therein while FIGS. 6-23 describe a first embodiment of the device with illustrative incisions. FIGS. 24-38 describe a second embodiment of the vascular device with additional illustrative incisions. FIGS. 39-53 describe a third embodiment with delivery sheaths and a helical anchor for tying tissue together. FIGS. 54-69 provide a fourth embodiment localizing a plurality of pledgets and sutures made of biodegradable materials secured together with a knot to allow re-access. FIG. 70 provides for different techniques. Components described below within the embodiments may be interchanged, removed, or added within or to one another to come up with derivatives of the device which are within the scope of the present disclosure.

Turning to FIG. 1, a front view schematic representation of an illustrative human venous circulatory system of a patient 100 with a guidewire 102 routed from a femoral vein 104 into a right atrium 106 in accordance with one aspect of the present disclosure is provided. The guidewire 102 may permit a continuous presence whereby multiple tools may be exchanged. For example, these tools may provide placement of pre-closure sutures, subsequent controlled atrial septostomy by a retractable blade, and delivery of a large bore catheter or other medical device to the left atrium.

Initially, as shown, a vascular introduction sheath 112 may be inserted into the right femoral vein 104 via a percutaneous puncture or incision. Alternatively, the vascular introduction sheath 112 may be placed into a non-femoral site such as a jugular vein, subclavian artery, subclavian vein, or brachial artery and vein. Other approaches or access sites may include an approach of the opposite leg from the therapy catheter.

The guidewire 102 may be inserted through a vascular introduction sheath 112 and routed cranially up the inferior vena cava 110 to the right atrium 106, one of the chambers of the heart 108. In this illustration, the left anatomical side of the patient 100 is toward the right. The guidewire 102 may be placed so that it is used to direct therapeutic or diagnostic catheters into a region of the heart 108.

The venous circulation, through which the guidewire 102 has been routed, may generally be at a lower pressure between 0 and 20 mm Hg than is the systemic circulation, of which the descending aorta is a part of. The pressure within the systemic circulation may range from 60 to over 300 mm Hg depending on the level of hypertension or hypotension existent in the patient 100. By accessing the heart 108 through the venous circulation, at the femoral vein 104, the chance of hemorrhage from the catheter insertion site may be minimized.

FIG. 2 is a front view schematic representation of the illustrative human venous circulatory system of the patient 100 with an exemplary vascular device 200 advanced into a right atrium 106 in accordance with one aspect of the present disclosure. The view is a frontal illustration, looking posteriorly from the anterior side of the patient 100. The vascular introduction sheath 112 of FIG. 1 has been removed from the right femoral vein 104 and a vascular device 200 has been inserted into the venous circulation over the guidewire 102. The device 200 may be routed through the inferior vena cava 110 into the right atrium 106 of the heart 108 through the same guidewire 102 used by the introduction sheath.

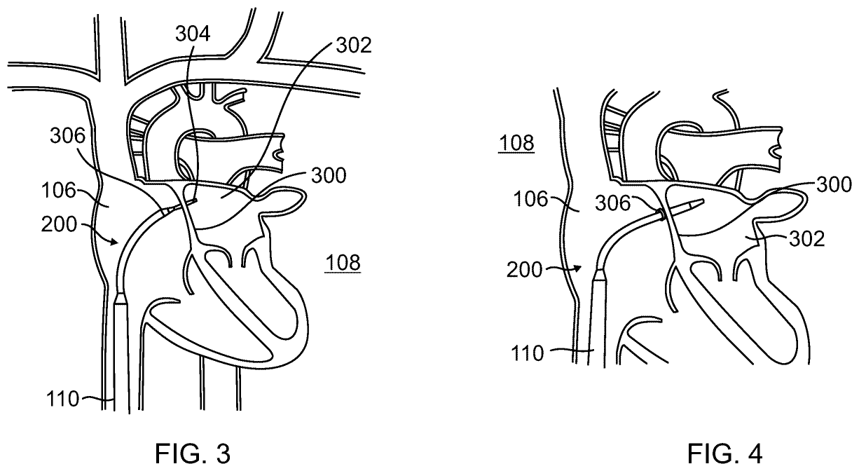

With reference to FIG. 3, a cross-sectional illustration of the heart 108 with the exemplary vascular device 200 positioned at an atrial septum 300 and a septal penetrator 304 advanced across the atrial septum 300 into a left atrium 302 in accordance with one aspect of the present disclosure is shown. An ascending aorta, aortic valve, pulmonary artery, and pulmonary valve have been removed from this illustration for clarity and to show the atrial septum 300. The body of the vascular device 200, substantially located within the right atrium 106, is shown with its long axis perpendicular to the atrial septum 300. The proximal end of the vascular device 200 is shown resident within the inferior vena cava 110. A septal penetrator 304 is shown extended through a puncture 306 in the atrial septum 300 and is routed into the left atrium 302 on a distal end of the vascular device 200.

The septal penetrator 304 may be a needle or axially elongate structure with a sharp, pointed distal end. The septal penetrator 304 may be resident within the guidewire 102, with the penetrator 304 being removable. The septal penetrator 304 may be actuated at the proximal end of the vascular device 200 through a control mechanism such as a button, lever, handle, or trigger which may be affixed, permanently or removably by way of a linkage, pusher rod, electrical bus, or the like that runs the length of the device 200.

In operation, the septal penetrator 304 through a wall of the left atrium 302 opposite the atrial septum 300 may be guided and advanced using fluoroscopy, magnetic resonance imaging (MRI), ultrasound, or the like. Care may be taken not to inadvertently pierce the aorta through the penetrator 304 in the region upstream or anatomically proximal to an aortic arch of the patient 100. The distal portion of the vascular device 200 may be bent, deflected, or articulated through an angle of between 30 and 120 degrees to achieve approximate perpendicularity with the atrial septum 300.

The septal penetrator 304 may be solid, it may be hollow like a hypodermic needle, or it may have a “U” or “C”-shaped cross-section. The center or core of a hollow “C” or “U”-shaped septal penetrator 304 may be filled with a guidewire or other core element to prevent incorrect tissue penetration. The septal penetrator 304 may be rigid or it may be flexible but retain column strength. Such flexible configurations may include cutouts in the wall of the penetrator 304 or guidewire-like construction. The septal penetrator 304 may be initially straight or it may be initially curved. The septal penetrator 304 may be fabricated from shape memory material such as nitinol and heat treated to cause curving once the material is heated from martensitic to austenitic temperatures. Such heating may be performed using electrical heating, hot water injection, or the like. The septal penetrator may utilize energy to facilitate puncture of the septal tissue such as RF (Radio Frequency).

Referring to FIG. 4, a cross-sectional illustration of the heart 108 with the exemplary vascular device 200 advanced into the left atrium 302 across the atrial septum 300 and the septal penetrator withdrawn in accordance with one aspect of the present disclosure is shown. The vascular device 200 having expanded at a distal portion has advanced across the atrial septum 300 from the right atrium 106 and into the left atrium 302 through the puncture 306. Through this, the distal portion of the vascular device 200 may provide placement of pre-closure sutures or other apparatuses.

The proximal region, or body, of the vascular device 200 has advanced so that the proximal region is located not only in the inferior vena cava 110 but also within the right atrium 106. This may be guided by fluoroscopy, magnetic resonance imaging (MRI), ultrasound, or the like, which was described earlier.

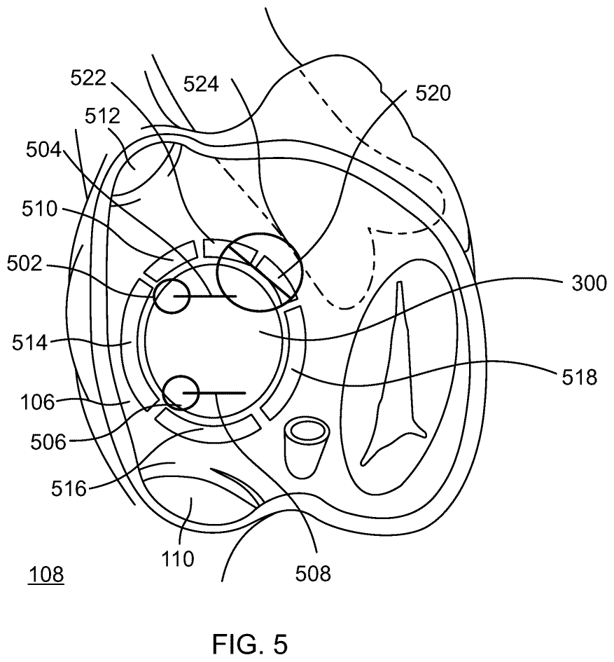

FIG. 5 is a cross-sectional illustration of the heart 108 showing illustrative initial incisions 502 or 506 and followed by lengthening them to a user selected/controlled length in accordance with one aspect of the present disclosure. A cross-sectional view of the heart 108 is shown viewed from a right atrial side. The atrial septum 300 may be surrounded by the superior vena cava rim 510 of a superior vena cava 512, posterior rim 514, inferior vena cava rim 516 of the inferior vena cava 110, atrioventricular valve rim 518, aortic rim 520, and superior rim 522. The vascular device 200 may create a user defined adjustably controlled atrial septostomy by a retractable cutting implement, in conjunction with suture-mediated closure created by the atrial septostomy.

After the puncture is made into the atrial septum 300 by the septal penetrator at an ideal location, the distal end of the vascular device 200 may be inserted through the tissue plane. An initial incision 502 or 506 on the atrial septal wall may be made and then subsequently lengthened to a specified desired amount 504 or 508 appropriate to allow for a therapeutic instrument. Additionally, positional control and visualization may enable the user to avoid areas 524 of the anatomy that are not desirable to disturb such as the aortic rim 520 or superior rim 522 or puncturing outside the atrium, or cutting through myocardium

The target of the tissue incision 502 or 506 may be on the atrial septum 300 in such a location to allow access to the desired target of therapy such as the mitral valve. In one example, the incision length or size may be set to accommodate the procedural instrumentation or device without further damaging the tissue plane. The tissue when cut to a specific length that is large enough may facilitate no ripping of the tissue beyond the desired incision length. This may be accomplished by having the perimeter of the incision 502 or 506 along with the desired amount 504 or 508 match the circumference of the therapeutic instrument. In one embodiment, creating a cut that is slightly larger than the subsequent therapy catheter may enable more flexibility to the user. In a typical septal puncture, if the initial puncture site is not adequate, the user may need to retract the catheter, re-puncture, and re-dilate with the risk of tearing the tissue.

The length may also be adjusted to account for stretching of the tissue plane. The tissue rim surrounding the atrial fossa may be used or referenced to begin or limit the incision 502 or 506 of the tissue. Extending the incision 502 or 506 beyond or outside the rim of the fossa may require more force or energy to create and therefor utilized as a feedback loop to determine the location of the incision 502 or 506.

In one example, which will be shown below, the vascular device 200 may have one cutting arm and a centering puncture member. This configuration may have the operator rotate the tool to create a slit in a desired direction. The edge of the fossa of the heart 108 may be used as the starting point as this point may be easier to puncture through first and then rotate the cutting member to cut along a direction of choice. Alternatively, two symmetrical cutting arms extending from center (with or without a centering puncture face) may be used. This configuration may allow the physician to puncture a known height or position which may allow them to be sure that the center of the cut may be at the position as they intend, as the cut is symmetric.

Turning to FIG. 6, which discloses a first embodiment, an illustrative side view of the exemplary vascular device 200 in accordance with one aspect of the present disclosure is provided. The vascular device 200 may have a distal region, which may be the tip or anchor, expandable from a body of the device 200. A length of sheath tubing 606 may extend the body to a sheath hub 608. The tubing 606 may be substantially curved near its distal end to provide deflection of catheters in a direction approximately 180 degrees from the exit path of the guidewire 102. As illustrated, the sheath hub 608 may be a tri port hub 608. The hub 608 may be configured, in other embodiments, with less or more ports. Multiple instruments may be inserted and retracted from the hub 608, which will be shown below.

Broadly described, the vascular device 200 may be used to place sutures into the atrial septum, either before or after creating a controlled atrial septostomy through instruments placed into the hub 608. Other medical devices may gain entry and location to the left atrium, with the ability to subsequently close that ASD. Advantageously, the way the ASD is closed may permit future tissue crossings in the event that is necessary for a subsequent catheter based procedure.

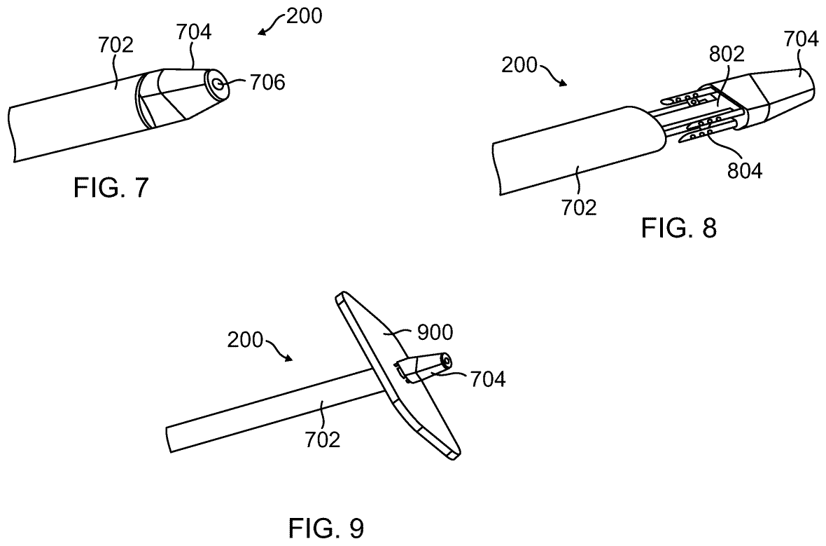

Turning to FIG. 7, an isometric view of the distal end of the exemplary vascular device 200 in a low profile condition in accordance with one aspect of the present disclosure is provided. The device 200 may permit placement of the atrial septostomy closure sutures prior to introduction of the cutting implement, that is, a blade septostomy. The entire device 200 may run over the guidewire, which was disposed to the atrial septum through the right atrium.

The vascular device 200 described herein may facilitate creation of a controlled and or adjustable size and position an access incision in a tissue plane such as an atrial septum allowing for a therapeutic instrument or catheter to easily perform a controlled closure of the incision after the procedure is complete. The closure may be tailored to be sealed hemodynamically, or conversely allow for certain amounts of flow. The therapeutic instrument may perform diagnosis and therapeutic intervention to correct atrial fibrillation, perform mitral valve repair, correct septal defects, or perform implantation of a cardiac prosthesis, for example.

The vascular device 200 at a distal end may include a catheter shaft 702 or body, anchor 704, and guidewire lumen 706. These components may be made of, for example, a polymeric material. Elastomeric materials may be used to construct the catheter shaft 702 to maximize flexibility. These materials may be used to construct an inner and outer wall of the shaft 702. Reinforcing structures within the device 200 may be made from metals, such as stainless steel, titanium, or the like. In this embodiment, the reinforcement structure is malleable but retains sufficient force to overcome any forces imparted on it.

The catheter shaft 702, or delivery catheter, may have a body that is tubular in structure. The shaft 702 may, in one embodiment, have a circular cross-section for housing components. These components may extend towards a proximal end of the vascular device 200. Left atrial appendage implants described below may be radially collapsible during delivery through the shaft 702. In one embodiment, the implants may be delivered through 14 French or larger catheters with a radially expandable delivery sheath.

Continuing with FIG. 7, the anchor 704 of the vascular device 200 may be delivered to the atrial septum and extendable from the catheter shaft 702. The anchor 704 and catheter shaft 702 may incorporate components which perform specific functions that enable the delivery of sutures across tissue from a remote location. The anchor 704, for purposes of this embodiment, may be in a cone-shape. Alternatively, the anchor 704 may have a shape that is, but is not limited to, funneled, rounded, tipped, or peaked. The anchor 704 may have a beveled edge which may conform to the atrial septum.

Traveling or within the catheter shaft 702 and anchor 704 of the vascular device 200 may be the guidewire lumen 706. The lumen 706 may permit advancement and delivery of the vascular device 200 over the previously placed guidewire. The guidewire, which was described earlier, may enter into the patient through a femoral vein up into the inferior vena cava and into the right atrium. The septal penetrator for placing the initial puncture may be resident within the guidewire, which may be removable therefrom.

FIG. 8 is an isometric view of the distal end of the exemplary vascular device 200 viewed from a different perspective with a portion of the device 200 advanced revealing components used to puncture tissue and pass sutures in accordance with one aspect of the present disclosure. The anchor 704, having a cone-shape, may be advanced within the heart of the patient from a tip of the catheter shaft 702. The amount of distance that the anchor 704 advances may depend on the thickness of the tissue it is crossing and the size of the chamber that it is entering.

The vascular device 200 may be designed to permit or restrict a varying amount of the anchor 704 to travel. The travel may be as small as a few millimeters to upwards of several centimeters. For purposes of this disclosure, it is assumed that the anchor 704 may cross the interatrial septum from the right atrium into the left atrium and the catheter shaft 702 may remain on the right side of the heart within the right atrium. The anchor 704 may be extended such that needles 804 of the anchor 704 are allowed to be hooked or grabbed within the left atrium 304.

Once the anchor 704 has been advanced, the needles 804 may be exposed. In one embodiment, as shown, four needles 804 may be removably coupled into the anchor 704. The needles 804 may be rearward facing. For example, the needles 804 may extend towards the catheter shaft 702, or body, of the vascular device 200 when the anchor 704 has extended into the left atrium. The face of the catheter shaft 702 and anchor 704 may be angled a prescribed amount to permit a more orthogonal contact with the tissue which should promote a more stable interface between the tissue and catheter shaft 702.

An advancement shaft 802 for the anchor 704 of the vascular device 200 may be rectangular in shape and travel through a corresponding rectangular-shaped lumen within the catheter shaft 702 in order to maintain a precise rotational alignment with the catheter shaft 702. Typically, the alignment between the anchor 704 and catheter shaft 702 is maintained as it permits device functionality. The advancement shaft 802 may be extended and retracted at a proximal end of the vascular device 200, which as shown above may be located at the percutaneous puncture or incision point.

Referring to FIG. 9, an isometric view of the distal end of the exemplary vascular device 200 with the portion of the device 200 advanced through the tissue 900 of the atrial septum in accordance with one aspect of the present disclosure is provided. The vascular device 200 has been placed through a representative section of tissue 900. The anchor 704, having a cone-shape, has been advanced across the puncture site of the tissue 900 which was performed earlier by the septal penetrator. The catheter shaft 702 may be larger than the puncture created by the septal penetrator, thus precluding the catheter shaft 702 from entering into the tissue 900. This may allow for the tissue to be tight or secured around the narrow aspect of the apparatus.

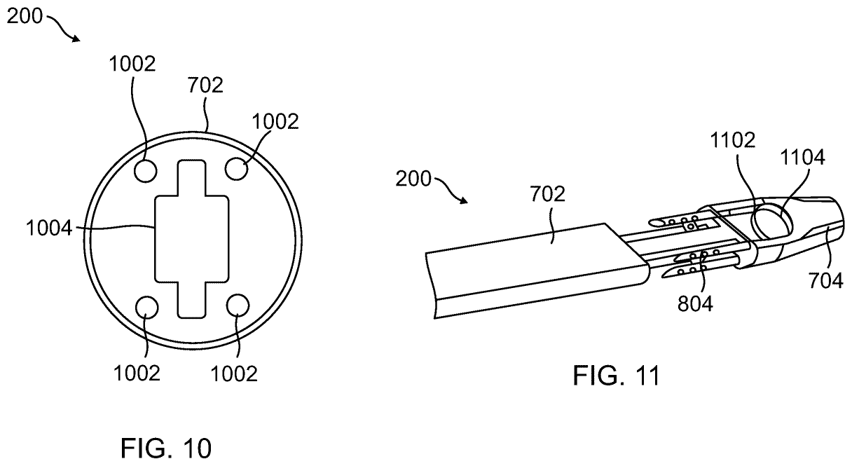

FIG. 10 is a cross sectional view of the exemplary vascular device 200 having an illustrative lumen configuration in accordance with one aspect of the present disclosure. The catheter shaft 702 may include several channels or lumens that permit certain components to pass therethrough. As an example, four lumens 1002 having equal diameters may permit snares to grab, hook or loop the needles removably coupled to the anchor with tethered sutures to pass through them. The central rectangular-shaped channel 1004 may permit the rectangular-shaped advancement shaft of the anchor to travel through it. The configuration may maintain an alignment of the four needles and cutting implements, which will be shown later, within the catheter shaft 702.

Different configuration to the cross section of the vascular device 200 may be implemented depending on suture placements. For example, more than four lumens 1002 may be channeled through the catheter shaft 702 with each being equidistant from the center. Advantageously, the lumens 1002 may provide a proper spacing to the initial puncture such that sutures management may be had. Punctures used to capture the needles into the lumens 1002 may be placed such that no unnecessary tearing of tissue is made yet still proper suture placement is performed. The shown configuration may allow for two sutures through four needles, but other configurations may exist and are within the scope of the present disclosure. In one embodiment, apertures may radially surround the central lumen where the user may selectively advance any number of needles/sutures through. The sutures may be loaded from the proximal end in any configuration the user has chosen.

FIG. 11 is an isometric, sectioned view of the distal end of the exemplary vascular device 200 revealing the inner geometry of components therein in accordance with one aspect of the present disclosure. For illustrative purposes, a section of the anchor 704 has been removed permitting a view of a bundled suture 1102 that is stowed in a recess channel 1104 of the cone-shaped anchor 704.

Ends of the suture bundles 1102 may be coupled to two needles 804 on opposite ends. Two suture bundles 1102, as shown, may have four needles 804. The suture bundles 1104 may be placed on opposite sides of one another delineated, or separated, by the advancement shaft. The needles 804 on both sides may be engaged simultaneously with the recess channel 1104 unspooling both suture bundles 1102. The recess channel 1104 may rotate for two different bundled sutures 1102 while the needles 804 are being drawn into the catheter shaft 702.

The recess channel 1104 may be shaped to permit the unspooling and release of the suture bundles 1102. In one example, sutures of the suture bundles 1102 may be spooled into the recess channel 1104 which may be held taught within the anchor 704. When the needles tethered to the suture bundles 1102 are pulled, the suture bundles 1102 may be unspooled. The sutures of the suture bundles 1102 may be released from the anchor 704 after the suture bundles 1102 are pulled a predetermined amount by the needles 804 which are drawn into the catheter shaft 702. This amount may be, for example, a couple of centimeters.

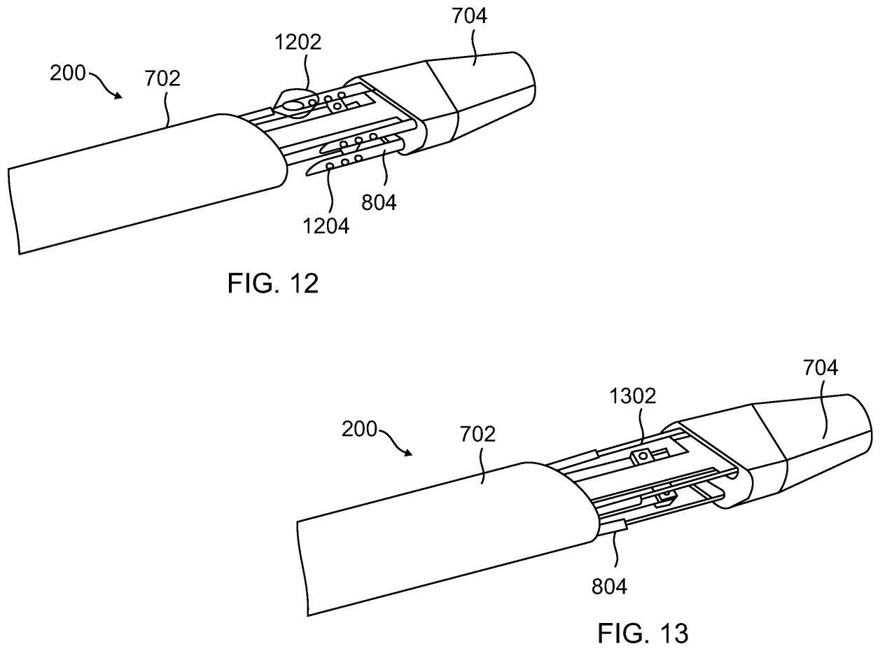

With reference to FIG. 12, an isometric view of the distal end of the exemplary vascular device 200 revealing snares 1202 that have been advanced out of a body of the vascular device 200 in accordance with one aspect of the present disclosure is provided. Four snares 1202 may be used to grab, hook or loop the suture bundles with each suture bundle having on opposite ends needles 804 for snaring. Small punctures may be made for the snares 1202 to pass through the tissue, or the snares 1202 themselves may have a tip capable of puncturing the tissue. The snares 1202 may be placed through the catheter shaft 702 at a proximal end and out of the previously described lumens. Once the needles 804 have been captured, the snares 1202 may be retracted through the catheter shaft 702.

In one example, recesses 1204 placed within the needles 804 may be used by the snares 1202. These recesses 1204 may be slanted and directed towards the anchor 704 of the vascular device 200. When the snares 1202 are pushed through the catheter shaft 702, a hook of the snare 1202 may be pulled and tethered against the recess 1204.

A mechanism may be used at a proximal end of the catheter shaft 702 to push multiple snares 1202 therethrough simultaneously. The snares 1202 may grab and then pull the needles 804 through the tissue at the same time. In an alternative embodiment, the snares 1202 may be individually pushed into the shaft 702 to capture or hook a single needle 804 at one time through its recess 1204. In one embodiment, two snares 1202 may work in tandem to pull two corresponding needles 804 through the shaft 702. The two needles 804 may be connected to opposite sides of the suture bundle. The needles 804 may be trimmed from the suture after being pulled into the shaft 702.

Snares 1202 may be made of a variety of materials. For example, the snares 1202 may be made of a radiopaque platinum coil and tip for enhanced visibility. The snare 1202 may include a helical loop design for a smaller profile yet a longer reach than right-angle loops. A durable cobalt chromium loop may add strength and retain its shape. The snare 1202 may have a loop that varies in size: 1 mm, 2 mm and 3 mm loop diameters for clinical versatility.

FIG. 13 is an isometric view of the distal end of the exemplary vascular device 200 after four suture needles 804 and suture ends have been passed through the tissue 900, snared and pulled through the length of the device 200 in accordance with one aspect of the present disclosure. The catheter shaft 702 of the vascular device 200 may receive the needles 804 after being pulled by the snares. From the bundled sutures with the pull of the needles 804 coupled on opposite ends, sutures 1302 may be unspooled from the recess channel and tensioned.

The suture 1302, in one embodiment, may be made of finely woven nylon material. Other materials may be used such as, but not limited to, polypropylene, silk or polyester. The sutures 1302 may be made of a sturdy, but bendable material. The sutures 1302 may be in a “U” or “C” shape. The sutures 1302 may be soaked in a sterile mineral oil immediately prior to its use. The edges of the suture 1302 may be sutured easily to margins of an incision in the atrial septum. The suture may be made of a high temperature resistant material to prevent damage if it is in contact with the cutting implement.

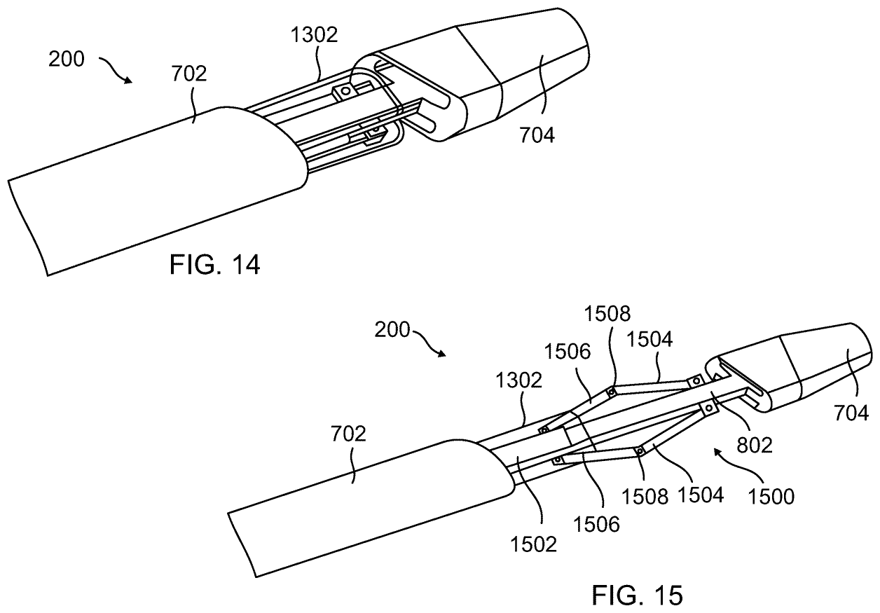

FIG. 14 is an isometric view of the distal end of the exemplary vascular device 200 after the suture 1302 has been pulled completely through the length of the device 200 and is now pulled taught against the tissue in accordance with one aspect of the present disclosure. The sutures 1302 may be fully unspooled from the recess of the anchor 704. The ends of the suture 1302 may be available to the user through the proximal end of the catheter shaft 702 of the vascular device 200. In operation, the suture lines may be slackened or tightened according to a user's need at a particular time.

With reference now to FIG. 15, an isometric view of the distal end of the vascular device 200 after a cutting implement 1500 has been partially advanced revealing the cutting elements 1506 in accordance with one aspect of the present disclosure is provided. Once the sutures 1302 are pulled against the tissue, such that they may not be cut, the anchor 704, having the cone-shape, may be further advanced into the left atrium through the advancement shaft 802. The shaft 802 may be extend through the catheter shaft 702 of the vascular device 200 towards and through the puncture in the atrial septum. The advancement shaft 802 may be extended through the proximal end of the vascular device 200.

By advancing the shaft 802 through the tissue of the atrial septum, a second, rectangular-shaped telescoping cutting implement 1500 may be sent through the rectangular-shaped channel of the catheter shaft 702. The cutting implement 1500 may include an expansion actuating shaft 1502 that may telescope over the advancement shaft 802 for the anchor 704. That is, the expansion actuating shaft 1502 may slide over the advancement shaft 802 of the anchor 704.

The expansion actuating shaft 1502 may be advanced through the puncture and be located within the left atrium extending the cutting implement 1500. When the expansion actuating shaft 1502 is pushed forward, the cutting implement 1500 with a linkage system 1504 is exposed. A distal end of the cutting implement 1500 may be temporarily locked to a top portion of the advancement shaft 802 of the anchor 704 while a proximal end of the cutting implement 1500 may be connected to a lower portion of the expansion actuating shaft 1502.

The linkage system 1504 may bow outward and expand the cutting elements 1506 to a length much greater than the diameter of the vascular device 200 when the anchor 704 is retracted and the expansion actuating shaft 1502 is held in place. The linkage system 1504 may bend at symmetrical points 1508 when the advancement shaft 802 is retracted. The cutting elements 1506, as shown, are positioned behind the anchor 704 facing towards the catheter shaft 702. In operation, the linkage system 1504 may remove any potential to cut the sutures 1302, which are parallel thereto, as the cutting elements 1506 are expanded.

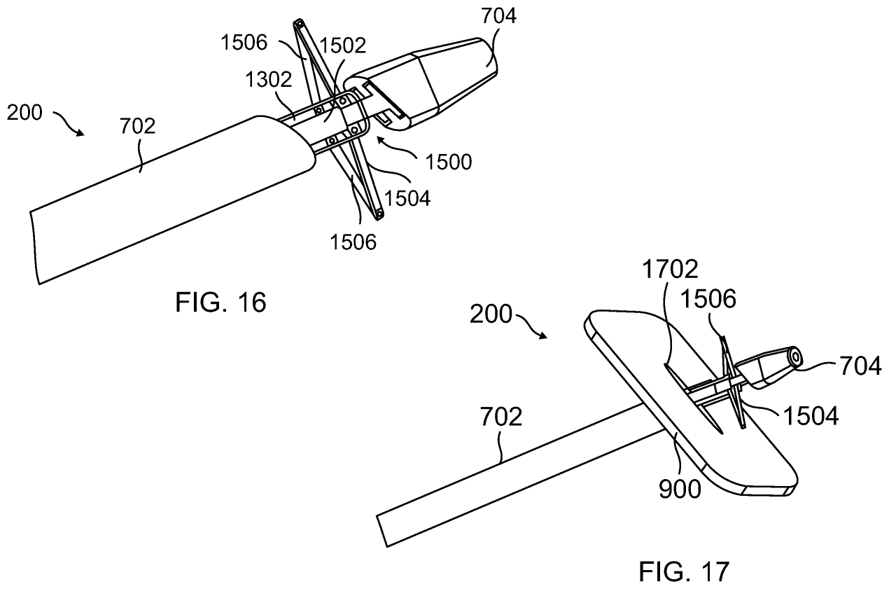

FIG. 16 is an isometric view of the distal end of the vascular device 200 with the cutting implement 1500 expanded in accordance with one aspect of the present disclosure. The anchor 704, in one configuration, has been retracted covering the advancement shaft. When this is performed, the top of the cutting implement 1500 is held and lowered with the anchor 704 and the expansion actuating shaft 1502 holds a bottom portion of the cutting implement 1500 in place. This may cause a bend in the cutting implement 1500 within the linkage system 1504 and expose the cutting elements 1506. The cutting elements 1506 may be perpendicular to the shafts, such that the cutting elements 1506 expand past a diameter of the catheter shaft 702.

The parallel alignment of the cutting elements 1506 relative to the sutures 1302 may be made so that the cutting elements 1506 do not inadvertently cut the sutures 1302. Multiple cuts or incisions may be made by cutting elements 1506 by slicing through the tissue and pulling through the cutting elements 1506 back to the left atrium. The processes may be repeated depending on the number of incisions needed. Accordingly, the number and location of sutures/needles may vary depending on the size and number of incisions made.

When completed, the cutting elements 1506 may be retracted by advancing the anchor 704. The linkage system 1504 may be collapsed through this advancement and the system 1504 may condense into a narrow channel without the cutting elements 1506 exposed. The lock coupling the top of the advancement shaft and the cutting implement 1500 may be removed. The cutting implement 1500 may then be pulled through the catheter shaft 702 without moving the anchor 704. After removing the cutting implement 1500, the vascular device may be removed leaving the sutures 1302 in place.

Other technique or devices may be used to expand and collapse the cutting elements 1506 such that no tissue is inadvertently cut when the anchor 704 of the vascular device 200 is being used in the left atrium. The linkage system 1504, allowing the cutting elements 1506 to be used, may come in a variety of forms and is not necessarily limited to that shown in this embodiment. For example, the linkage system 1504 may entirely reside on the expansion actuating shaft 1502 whereby mechanisms on the proximal end may be used to expand and collapse the cutting elements 1506 without the need to retract the anchor 704. This may use a separate knob, pull-wire, or the like to expand and collapse the cutting elements 1506. Other variations may exist and are within the scope of the present disclosure.

With reference now to FIG. 17, an isometric view of the exemplary vascular device 200 positioned to cut the tissue 900 in accordance with one aspect of the present disclosure is provided. The vascular device 200 may be angled at a prescribed amount to permit a more orthogonal contact with the tissue 900. This may promote a more stable interface between the tissue 900 and catheter shaft 702.

The distal end of the catheter shaft 702 may be angled to conform to the tissue 900. The shaft 702 typically does not go through the initial puncture or the slit 1702 created by the cutting elements 1506. The anchor 704 of the vascular device 200, however, may extend through the puncture and into the left atrium. The linkage system 1504 may be expanded and the cutting elements 1506, which may be in the form blades, may be used to cut a slit 1702 through the tissue 900. The length of the slit 1702 may be controlled by how much the linkage system 1504 is expanded. The vascular device 200 may be rotated to produce other slits 1702 or combinations of slits 1702.

In operation, the anchor 704 of the vascular device 200 may advance through the initial puncture. Needles may extend towards the catheter shaft 702. The sutures may then be brought towards the tissue 900 from a backend before any incision 1702 is made. The sutures may then be managed after the incision 1702 and the therapeutic instrument has been used.

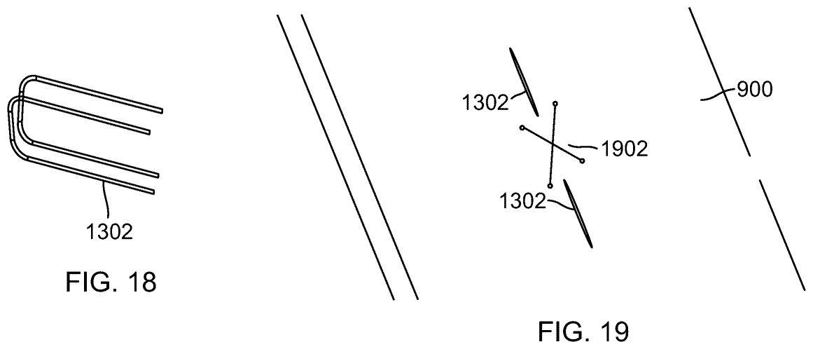

FIG. 18 is an isometric view of sutures 1302 that would be inserted into the tissue 900 in accordance with one aspect of the present disclosure. The two sutures 1302 may correspond to those that were unspooled from the recess of the anchor. After removing the vascular device, two lengths of sutures 1302 may remain in place behind the atrial wall that extends from the left atrium.

In addition, the guidewire previously used by the vascular device may also be left in place. The guidewire may be used by a larger bore device, such as a therapeutic instrument, that may now travel over the guidewire and gain easy access into the left atrium through the slit and run adjacent to the previously placed sutures. Once the large bore device has been removed, the free ends of the suture 1302 may be knotted and pushed towards the tissue to create a closing force. This may reduce the size of the cut or hole within the tissue, which the large bore device used. Advantageously, this may prevent or minimize the amount of hemodynamic communication between chambers and allow the user to leave behind a simple knot on the interatrial septum. Typically, the hole may close in the short term. In the long term, this may be used to permit a subsequent access into the left atrium if another catheter-based procedure or intervention is needed for the patient.

FIG. 19 is an isometric view of an incision after the tissue 900 is engaged with the suture 1302 in accordance with one aspect of the present disclosure. A close-up provides a view of the tissue 900 after a knot 1902 has been tied. A slit, which was created by the cutting implement has been cinched down in the middle by the knot 1902, which may reduce or eliminate an amount of hemodynamic flow and communication between chambers. For illustrative purposes the knot 1902 may be represented by a simple “X” configuration. Alternatively, the knot 1902 may be one of several different types of surgical knots that may be tied and pushed down the vascular device via a remote location.

In one embodiment, the knot 1902 may be formed and advanced with a knot pusher having a pusher rod fitted with a distal side port and severing member in the form of a sharpened outer sheath. The knot 1902 may hold an associated patch in place where an excess line may be trimmed by the shearing action of the pusher rod distal side port and the distal sharpened portion of the severing member. The excess line and other elements may be removed from the catheter.

Knot pushers that are known in the art include Edwards ThruPort knot pusher; Medline Endoscopic Pushers; Laparoscopic Knot Pushers by Cooper Surgical.

Arthrex offers several options: The Single-Hole Knot Pusher provides a simple method to advance sliding knots and half-hitches. This closed end knot pusher has a modified handle that provides an ergonomic feel. The distal tip has also been modified for easier advancement of slipknots and half-hitches. The 6th finger was designed to tie the surgeon's knots and allows the surgeon to apply and maintain tension to the first throw while advancing subsequent throws. The CrabClaw incorporates an opening jaw to allow intraarticular capture of suture.

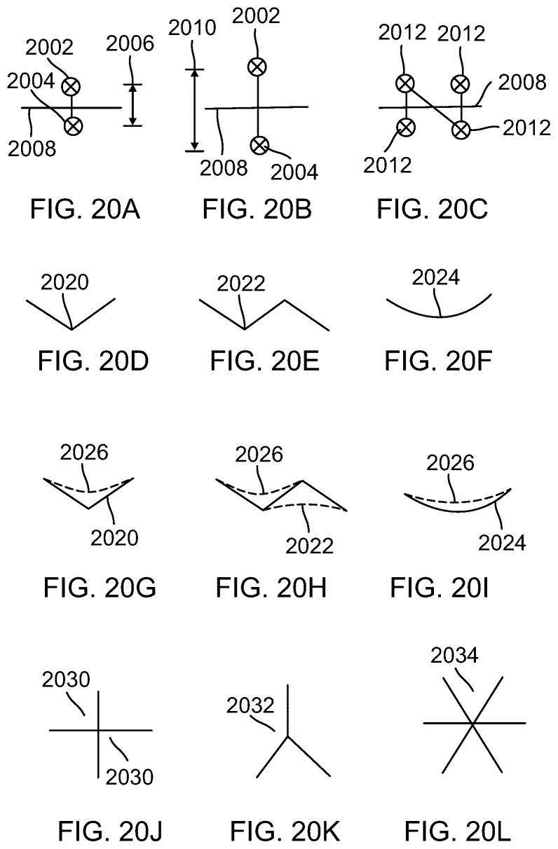

A simple incision with closure was described beforehand. Turning to FIGS. 20A-L, various illustrative cut patterns that may be created using the vascular device in the atrial septum which may be made from at least one cutting implement that may be rotated and used a single or multiple times in accordance with one aspect of the present disclosure are provided. The embodiments may create the various cutting shapes in the atrial septum. These shapes may be created with multiple cutting arms, or using one cutting arm that is rotated and used multiple times, such as the cutting implement shown above. The length of the incision may be controlled by the user through activation of the adjustable cutting implement. The incision may take a number of different geometries commonly used to facilitate both passage of the therapeutic device and closure of the tissue plane post therapy.

With reference to FIGS. 20A-20C, apposition of the tissue edges may be controlled by the nature of the location of the anchors or sutures 2002 and 2004. To increase overlap or tissue apposition, a first distance 2006 between the sutures 2002 and 2004 may be increased to a second distance 2010. Another method or technique may be to add additional suture locations or sutures 2012 at various and intersecting paths. The incision 2008 may be in the middle of the sutures 2002, 2004 and 2012.

The incision 2008 may take the form of many patterns such as a straight cut, v cut 2020, zig zag 2022, or crescent arc 2024, to name a few, and as shown in FIGS. 20D-F. These cuts may then be coupled together with other shapes yielding multiple flaps of tissue 2030, 2032, or 2034 shown in FIGS. 20J-L. The various configurations may be advantageous depending on the shape and size of the device needed to pass through and or the type or amount of closure that is desired post procedure. Some incision shapes such as FIGS. 20D-F may facilitate a tissue plane overlap 2026 upon closure due to the shape of the cut and the tension of the tissue before and during healing. This overlap may greatly aid the healing of the tissue edges together. That is, in FIGS. 20G-I what is shown is the tissue plane overlap 2026 from the different incision shapes 2020, 2022, and 2024.

The embodiments described herein manage the variables to control the closure of the incision 2008. The tissue edges may be managed by having control members in place before the incision 2008 is made. For example, and as shown above, having sutures 2002, 2004 and 2012 in place before incisions 2008 are made by the cutting implement may secure the tissue. In one embodiment, control features may be applied after the incision 2008 is made.

The tissue edge position and apposition of those edges relative to each other may be controlled to manage an amount of tissue overlap, apposition pressure, and amount of residual flow after a closer is applied. This control may be done by controlling where the suture lines are positioned relative to the incision. An example of this, is the distance of the suture lines farther away from the incision edge may cause more tissue bunching and/or tissue overlap. Increasing the number of tissue anchors and/or suture passing locations may increase the amount of tissue apposition along the length of the incision. The amount of tension or pressure applied to the suture tension lines may further affect the amount of closure on the incision 2008. These mechanisms may be managed in real-time and monitored under echo flow monitoring and/or fluoroscopy visualization.

In one embodiment, a mechanism may be used that would leave no long-term implant left in the patient. The mechanism may be designed to either seal the tissue, have the tissue heal in a sealed state, or have the tissue heal in a partially sealed state. The percentage of the hemostasis may be adjusted by varying the application of the mechanism. The mechanism may be designed to secure the tissue through various time points. These time points may correlate to various tissue healing cascade points such as time for tissue to coagulate, adhere, endothelialize, and scaring.

An absorbable body may be left that would facilitate the closure and be absorbed by the body over time. In one embodiment, the absorbable body may be removed from the body at a later point in time. The closure may be fully or partially engaged into the tissue plane near the incision 2008 before the incision 2008 is made. In another embodiment, applying the closure fully or partially engaged into the tissue plane may be near the incision 2008 after the incision is made. In one embodiment, the closure mechanism may be applied fully or partially engaged into the tissue plane near the incision 2008 after the incision 2008 and therapeutic instrument is removed from the incision 2008.

The cutting implements described herein may be used to control the condition of the tissue edges based on the method of creating the incision in the tissue. The methods of creating the incision may include, but are not limited to, a sharp blade made from durable material such as metal or ceramic, electrocautery techniques, RF energy, plasmajet vaporization, ultra-sonics, high voltage vaporization, controlled dilation, heat, cold, and others. A state of the cells on the edge of the cut surface may be controlled to optimize the desired healing cascade utilizing these various methods of incision creation.

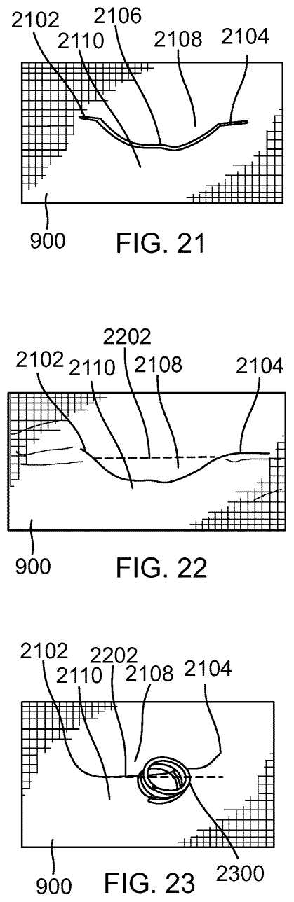

FIG. 21 is an isometric view of an illustrative cut pattern 2106 for incision to promote tissue edge apposition in accordance with one aspect of the present disclosure. A cut pattern 2106 may be defined by a first end 2102 and second end 2104. A cut pattern 2106 may be made into the tissue 900. The inside edge 2108 is separated from the outside edge 2110. This may be a cut pattern 2106 of a straight and arc combination for incision to promote tissue edge overlap and apposition.

FIG. 22 is an isometric view of the illustrative cut pattern for incision to promote tissue edge apposition, while under slight tension, demonstrating tissue edge control and overlap of edges with tension applied in accordance with one aspect of the present disclosure. This demonstrates overlap of the inside edge 2108 and outside edge 2110 with yield overlap 2202 in the tissue 900 caused by the cut pattern between the first end 2102 and second end 2104.

FIG. 23 is an isometric view of the cut patterns for incision to promote tissue edge apposition and a helical anchor 2300 controlling tissue edges in accordance with one aspect of the present disclosure. Tension may be applied in the direction of the ends 2102 and 2104 of the cut. The inside edge 2108 and outside edge 2110 with yield overlap 2202 in the tissue 900 may be secured with a helical anchor 2300, or the like.

Previously, a first embodiment of a vascular device was described. FIGS. 24-38 describe a second embodiment of a vascular device 2400 with additional illustrative incisions. It will be understood from the present disclosure that components with these embodiments may be interchanged, added or deleted based on a reasonable configuration. New embodiments using these modifications are within the scope of this disclosure.

Turning to FIG. 24, an isometric view of an illustrative expandable RF cutting implement 2410 with four expandable members 2416 in accordance with one aspect of the present disclosure is provided. The cutting implement 2410 may be between a catheter shaft and a distal anchor, as previously described. While four cutting members 2414 are shown, fewer or more may be used with each cutting member 2412 equidistant from one another.

The cutting implement 2410 may be expandable and collapsible through similar linkage systems described above. The cutting implement 2410 may be collapsed when advanced through the initial puncture and expanded after passing through the tissue. The cutting implement 2410 may have four expandable members 2414 connected to four cutting members 2412. The cutting members 2412 may be provided on a proximal end of the cutting implement 2410 such that the cutting implement is pulled back towards the tissue to make cuts.

The cutting members 2412 may extend radially from the center of the cutting implement 2410. The width of the cutting members may vary in width to change the incision length based on a French size of the delivery catheter. The cutting implement 2410 may also include a tip piercing device 2416 at a distal end of the cutting implement 2410. This may be used to puncture the tissue. The cutting members 2412 and tip piercing device 2416 may use mechanical or electrical energy. The mechanical or electrical energy may come from at least one of a blade, ceramic, electrocautery technique, RF, plasmajet vaporization, ultra-sonic, high voltage vaporization, controlled dilation, heat and cold. In one example, the cutting members 2412 and tip piercing device 2416 may both use mechanical energy. Alternatively, they may use both electrical energy. In yet another variation, the cutting members 2412 and tip piercing device 2416 may use different types of energy. The cutting implement 2410 along with its members 2412 and arms may radially expand in a controlled manner or plane such that they minimize or prevent the likelihood that the cutting implement 2410 inadvertently cuts or negatively impacts the previously placed closing sutures.

FIG. 25 is an isometric view of the exemplary vascular device 2400 with the expandable cutting implement 2410 having a tip for piercing the tissue 900 in accordance with one aspect of the present disclosure. A tip piercing device 2416 may be coupled to the cutting implement 2410 and may be pushed through the tissue 900 to create a puncture within the tissue 900. The puncture may be made through a mechanism on a proximal end allowing a physician to control the vascular device 2400. The cutting implement 2410 may be in a retracted state before advancing through the tissue 900.

A catheter shaft 2504, or delivery catheter, may house, but is not limited to, the cutting implement 2410 and anchor mechanisms 2502. The anchor mechanisms 2502 may be equidistant from the center of the catheter shaft 2504. While four anchor mechanisms 2502 are shown, fewer or more exist depending on a closure strategy of incisions made into the tissue 900.

The vascular device 2400, while not shown, may include visualization tools for determining a location of the device 2400 within the patient. In one embodiment, a sensor may be affixed to the device 2400 to determine a location and orientation of the catheter. Alternatively and/or additionally, an independent tracking system may be based on ultrasound, impedance or fluoroscopy tracking. In the case of impedance, electrical potential generated by electric field generators may be detected by the existing electrodes. In the case of fluoroscopy, electrode location may be detected by an image processing scheme that identifies and tracks the electrodes and/or opaque markers located on the device 2400.

FIG. 26 is an isometric view of the exemplary vascular device 2400 with the illustrative expandable cutting implement 2410 placed beyond the tissue 900 in accordance with one aspect of the present disclosure. The cutting implement 2410, in its retracted state, may be pushed through after the puncture is made to the tissue 900. The cutting implement 2410 may be sent through by an advancement shaft which may be controlled at a proximal end whereas the catheter shaft 2504 is not distributed therethrough.

After the cutting implement 2410 has been distributed into the left atrium, the cutting implement 2410 may be expanded. The expandable members 2414 may be radially extended from its center which spreads to a larger diameter than a diameter of the device 2400 itself.

An anchor mechanism 2502 within the catheter shaft 2504 may be used to push delivery mechanisms, which will be described below. The anchor mechanisms 2502 may be distributed within the catheter shaft 2504 and enclosed within lumens. The anchor mechanisms 2502 may surround the centralized cutting implement 2410 and be equidistant from one another.

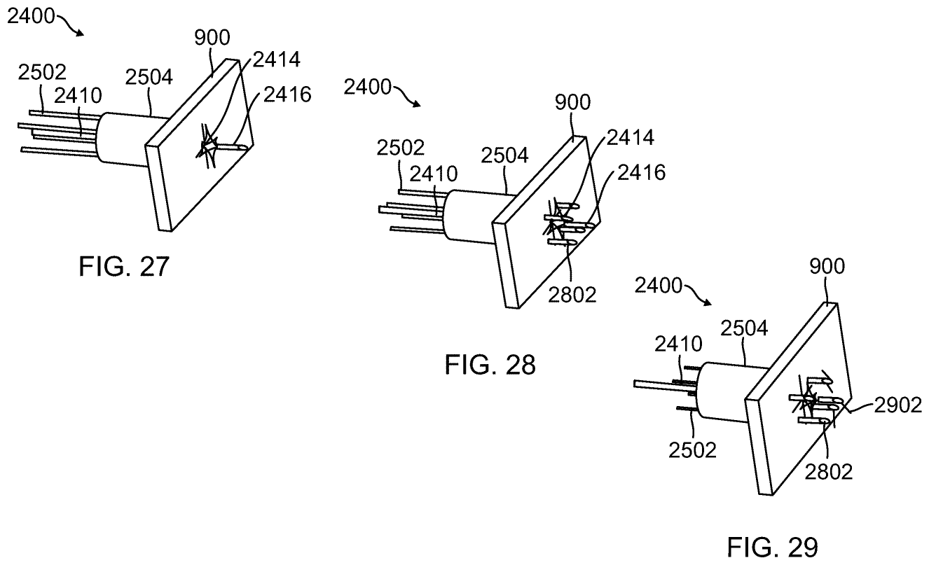

Referring to FIG. 27, an isometric view of the exemplary vascular device 2400 with the illustrative expandable cutting implement 2410 making incisions into the tissue 900 in accordance with one aspect of the present disclosure is provided. After the expandable members 2414 are extended, the advancement shaft may be pulled back, along with the tip piercing device 2416, toward the catheter shaft 2504 to make incisions in the tissue 900. These incisions may be made in a backplane of the left atrium. The cutting implement 2410 may be extended, rotated, and then retracted to make additional incisions into the tissue 900. During this time, the anchor mechanisms 2502 may be held stationary.

FIG. 28 is an isometric view of the exemplary vascular device 2400 with the illustrative expandable cutting implement 2410 advancing anchor delivery mechanisms 2502 in accordance with one aspect of the present disclosure. When the anchor mechanisms 2502 are pushed through the catheter shaft 2504, it may extend through the tissue 900 to the other side, that is, the left atrium. The anchor mechanisms 2502 may be coupled to a delivery mechanism 2802 that may be transferred through the tissue 900. The delivery mechanisms 2802 may have tissue piercing points.

Four delivery mechanisms 2802, coupled to four anchor mechanisms 2502, may pierce the tissue 900. Fewer or more combined structures may be present within the vascular device 2400. The delivery mechanisms 2802 may use similar energies to the expandable members 2414 and tip piercing device 2416. That is, combinations of mechanical and/or electrical energies may be used.

FIG. 29 is an isometric view of the exemplary vascular device 2400 with the illustrative expandable cutting implement 2410 with push anchors 2902 further inserted to advance the anchor mechanisms 2502 in accordance with one aspect of the present disclosure. The anchor mechanisms 2502 may advance the delivery mechanisms 2802 within the catheter shaft 2504 into and beyond the tissue 900, that is, within the left atrium. The push anchors 2902 may thereafter be deployed by the delivery mechanisms 2802. The push anchors 2902 may be used to secure the tissue 900, and its surrounding area. The anchors 2902 may be made from PLGA, PLLA, nylon, polyester, PEEK, or other biocompatible material. It should be noted that cutting implement 2410 may be advanced into the tissue 900 after the push anchors 2902 are set in place.

Other anchors may be used for securing the tissue 900. For example, tissue anchor lines may be utilized. These may include, but are not limited to sutures, toggles, helical structures, grabbing devices, inverting clips, expanding structures, mesh structures, stent-like structures, patch structures, clips, expandable valves, and suture knot configurations.

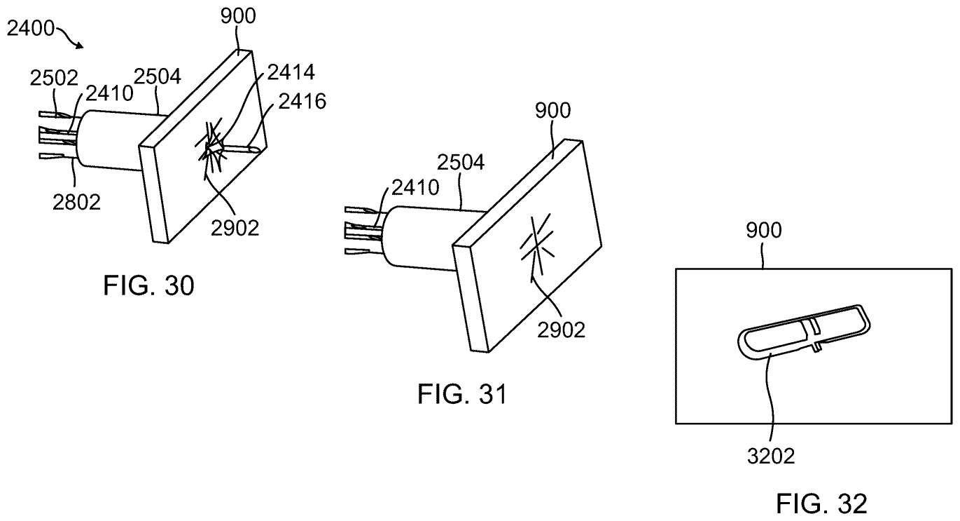

FIG. 30 is an isometric view of the exemplary vascular device 2400 having the delivery mechanisms 2802 removed in accordance with one aspect of the present disclosure. The anchor mechanism 2502 coupled to the delivery mechanisms 2802 may be pulled at a proximal end of the catheter shaft 2504. This may retract the delivery mechanism 2802 from the left atrium 302 into the right atrium 106. The cutting implement 2410 with its expandable members 2414 and tip piercing device 2416 may still be inserted into the left atrium of the patient. The anchors 2902 may be left against the tissue 900. These may be embedded therein.

FIG. 31 is an isometric view of the exemplary cutting implement 2410 removed from the tissue 900 leaving tissue anchors 2902 against it in accordance with one aspect of the present disclosure. The cutting implement 2410 may be removed through the catheter shaft 2504 of the vascular device 2400. The catheter shaft 2504 may still be placed in the right atrium of the patient at this time.

Referring to FIG. 32, an isometric view of an exemplary toggle 3202 within the tissue 900 in accordance with one aspect of the present disclosure is provided. The toggle 3202 may be pushed or advanced through the tissue longitudinally from the right atrium into the left atrium. The toggle 3202 may be shifted horizontally and secured on the septal wall to hold the tissue in place. The toggle 3202 may be made of similar materials to the anchor, which may be biodegradable. The presence of this closure apparatus or of others herein disclosed are of a size and location that they will not preclude another access procedure in the future.

Multiple cutting implements were described beforehand. These implements, as well as those described below, may use mechanical or RF energy. When using electrical energy, an amount of exposed metal may be minimized through insulation such that the exposed metal may only exist in the desired tissue cutting region of the tool. The smaller the amount of exposed metal, a better cutting effect on the tissue may be realized. Advantageously, this may use less power. To achieve the best cutting effect, the operator may ensure the cutting region is in good mechanical contact with the target tissue.

The initial septal puncture site and the septal cut may be performed using separate applications of energy. For example, using electrical energy, a first puncture may be performed using a first circuit and a larger cut may be achieved using a second circuit. If the first puncture is done separately from the cut, the operator may then rotate the cutting implement to align a cutting arm with a direction they intend to cut. If performing the second cut after the initial puncture by advancing the cutting tool from the right atrium to the left atrium, it may be beneficial to have the cutting regions of the initial puncture and the second larger cut overlap so there is no chance of a piece of tissue not being cut.

If the tool has symmetric cutting arms on either side of the center puncture element, the center of the overall cut may be at the intended puncture site, and not shifted in one direction. This is important for the success of a subsequent procedural step that may require being a certain distance above the target structures.

The following cutting implements may create a continuous cut form a center puncture site to the edge of the cut. The intent of these implements is to cut all the way from the center to the edge. The adjustability or expandability of the cutting implement may be achieved using a pull-wire/ring mechanism, such that the cutting implement may be compressed and bowed outward as the wire is pulled, which was described above. It may also be performed using a spring, or by using a shaped tool made of shape memory alloy.

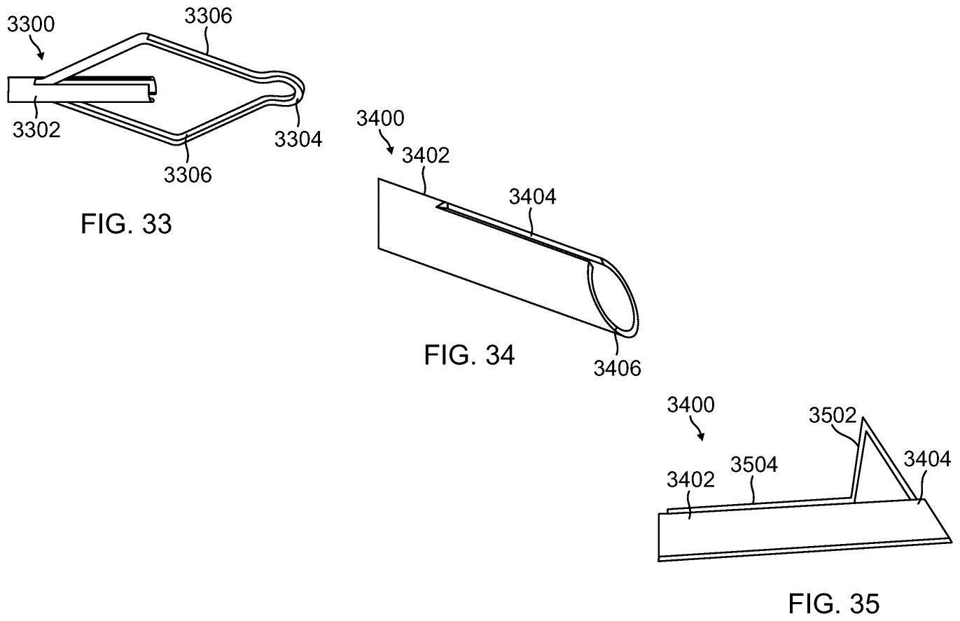

Turning now to FIG. 33, an isometric view of an illustrative cutting implement 3300 having an atraumatic tip 3304 deployed from an exemplary vascular device in accordance with one aspect of the present disclosure is provided. The cutting implement 3300 may be deployed from a distal end of a tubular member 3302 with electrical insulation selectively removed. This cutting implement 3300 may be fixed in size or adjustable through mechanism design.

The design may incorporate an atraumatic tip 3304 that may be used for finding the fossa ovalis or other desired target location. It also has cutting surfaces 3306 that may extend to both sides of the atraumatic tip 3304, symmetrically. In these types of embodiments, the initial septal puncture and slit creation may be performed in one motion with the same continuous cutting surface 3306, that is, they may be part of the same circuit for delivering energy, and they may also be on individual circuits. If the puncture/cutting energy is to be RF, microwave, or other electrical energy, insulation may be strategically removed from the metal structure. The amount of insulation removal, or alternatively metal exposure, may be varied to optimize performance. It may wrap entirely around the cutting arm, for example, or may be present in a narrow line along the length of the cutting surface arm. The goal may be to ensure the cutting surface being energize and in good contact with the tissue, with minimal direct communication to a blood pool.

FIG. 34 is an isometric view of an illustrative cutting implement 3400 having a slit 3404 in a sheath 3402 deployed from an exemplary vascular device in accordance with one aspect of the present disclosure. A cutting tool that incorporates one cutting arm may expand radially through the slit 3404 in the needle type sheath 3402, which will be shown below. In this embodiment, with only one cutting arm, the size of the arm may be fixed or adjustable through a mechanism, for example, using a pull-wire, spring, or the like. A distal portion 3406 of the cutting implement 3400 may be used for the initial puncture. Unlike the embodiments described above, the initial septal puncture site and slit creation are a part of separate parallel circuits, if electrical energy is used for cutting the tissue.

FIG. 35 is a side view of the illustrative cutting implement 3400 having a cutting element 3502 extending from the slit 3404 in the sheath 3402 in accordance with one aspect of the present disclosure. The expandable cutting arm 3504 is a part of a pull-wire that may run the length of the catheter and the puncture needle face is a part of another.

In one embodiment, the initial puncture needle may be entirely insulated, with only the distal region having exposed metal for energy delivery to the tissue. The expandable cutting element 3502, in the form of a radially extending arm, may have exposed metal circumferentially, or be mostly insulated with a thin line of exposed metal running along the cutting surface (or any number of patterns for exposing minimal surface area of the metal). It may be possible and more desirable for these two different cutting surfaces to be energized at the same time with one switch or through different switches on the handle end to allow for energy application at different times during the procedure. The benefit of only puncturing with the needle first is that it may create an anchor point in the tissue. Once this initial puncture is made, the operator may rotate the cutting implement 3400 until the expandable cutting element 3502 aligns with where they want the length of the cut to go.

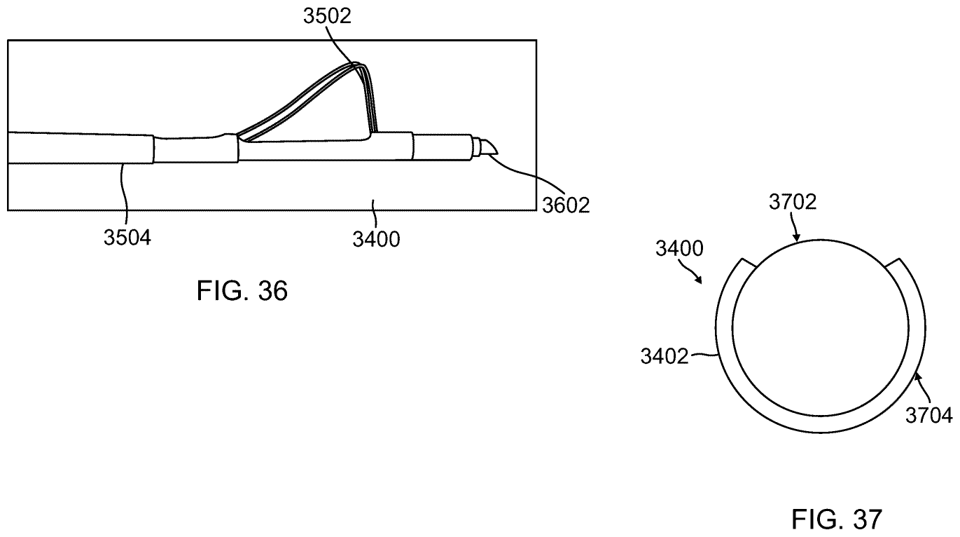

FIG. 36 is an isometric view of a distal end of the exemplary cutting implement 3400 in accordance with one aspect of the present disclosure. The expandable cutting arm 3504 for the cutting element 3502 may be a wire that runs the length of the catheter. A puncture needle 3602 may be part of another mechanism to be actuated. The cutting element 3502 may have exposed metal circumferentially, or be mostly insulated with a thin line of exposed metal running along the cutting surface. In operation, the expandable cutting arm 3504 may be pushed up and down to change the shape of the cutting element 3502.

The initial puncture needle 3602 may be entirely insulated, with only the distal region having exposed metal for energy delivery to the tissue. In one embodiment, the cutting implement 3400 may have two different cutting surfaces to be energized at the same time with one switch or through different switches on the handle end to allow for energy application at different times during the procedure. The benefit of puncturing with the needle 3602 first is that it may create an anchor point in the tissue. Once the initial puncture is made, the operator may rotate the tool until the expandable cutting arm aligns with where they want the length of the cut to go.

Furthermore, the cutting element 3502 may be retracted inward when the expandable cutting arm 3504 is pulled in a proximal direction. The cutting element 3502 may be retracted towards the center of the cutting implement 3400. The energy applied to the cutting element 3502 may be removed to prevent inadvertent cutting.

FIG. 37 is a cross-sectional illustration of the cutting implement 3400 in accordance with one aspect of the present disclosure. A schematic showing how the cutting arms of the embodiment may be made to allow exposed metal 3702 in specific areas to optimize the cutting performance of the tool and minimize the required power input is shown, which may use a thinnest possible insulation in order to ensure better tissue contact with the exposed metal surface. The sheath 3402 with no exposed metal using insulation 3704 is also provided. In this embodiment, the cutting element, or the cutting arm, is not used. Rather, it may use RF power for cutting.

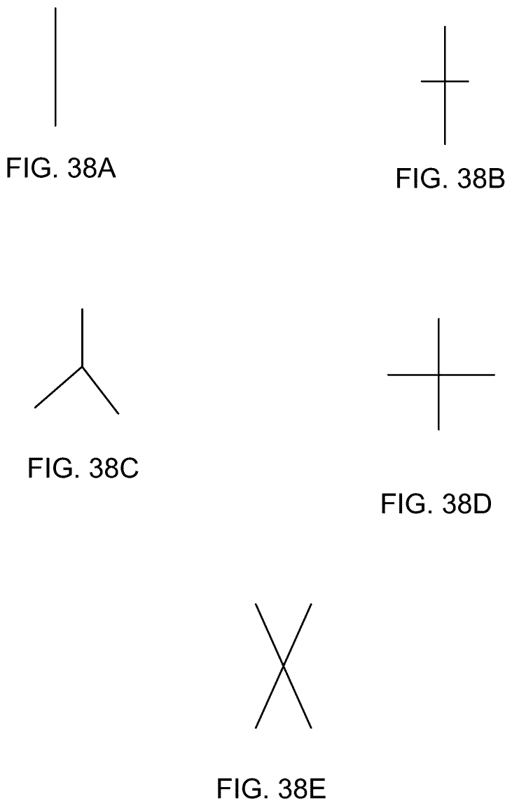

FIGS. 38A-E are schematics showing how the cutting implements may be used to optimize cutting performance and minimize power input in accordance with one aspect of the present disclosure. Various cutting shapes are shown that may be created by the embodiments through the interatrial septum. These shapes may be created with multiple cutting arms or using a single cutting arm that may be rotated and used multiple times.

FIGS. 39-53 describe a third embodiment with delivery sheaths and a helical anchor for tying tissue together. Components described below may be placed into a catheter shaft. The shaft internally may have a number of different lumens and channels for these components. In one embodiment, separate tools may be used for each of the components. These tools may follow along the guidewire that is in place. Techniques and/or devices are shown below to provide large bore transseptal access with subsequent atrial re-access.

Turning to FIG. 39, an isometric view of exemplary tissue 900 being crossed using an illustrative guidewire 102 in accordance with one aspect of the present disclosure is provided. The technique may initially begin with the guidewire 102 piercing or crossing into the tissue 900. The guidewire 102 may be brought in through the vascular introduction sheath which may be routed cranially up the inferior vena cava to the right atrium. The guidewire 102 may be placed to direct therapeutic or diagnostic catheters into a region of the heart.

FIG. 40 is an isometric view of suture anchors 4002 in delivery sheaths 4004 in accordance with one aspect of the present disclosure. The delivery sheaths 4004 may house other components in addition to the tissue anchors 4002. Using the guidewire, the delivery sheaths 4004 may be directed towards the tissue 900 of the atrial septum.

FIG. 41 is an isometric view of illustrative suture anchors 4002 in delivery sheaths 4004 about to penetrate the tissue 900 in accordance with one aspect of the present disclosure. The suture anchors 4002 may be placed at a first puncture point 4102 and a second puncture point 4104 which may be directed through a guidewire 102. Thereafter, they may be inserted into the tissue 900.

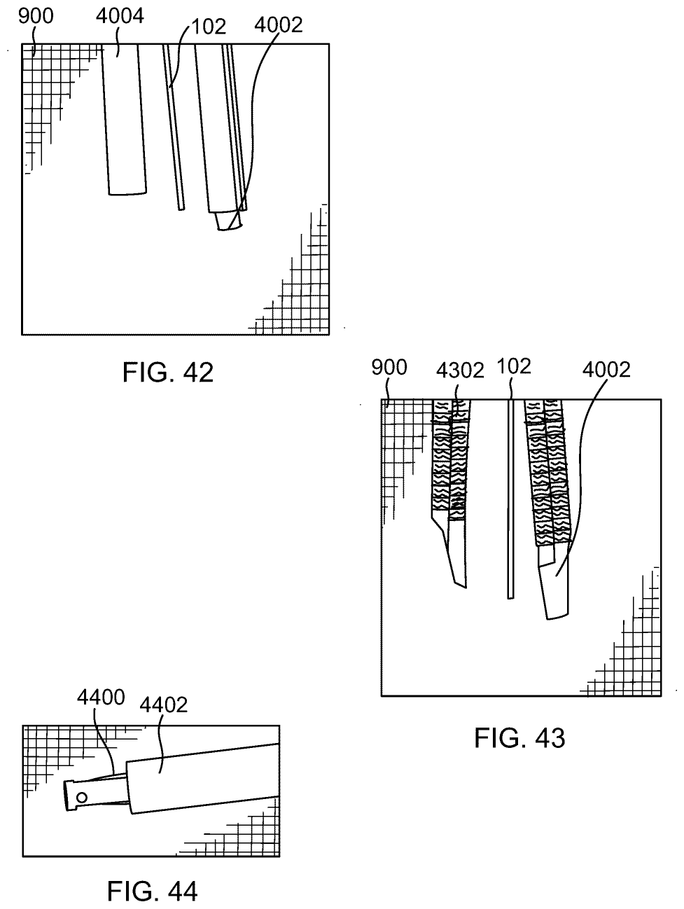

Referring to FIG. 42, an isometric view of the illustrative suture anchors 4002 in delivery sheaths 4004 engaging or penetrating the tissue 900 in accordance with one aspect of the present disclosure is provided. The suture anchors 4002 in delivery sheaths 4004 may engage or penetrate the tissue 900 near the guidewire 102.

FIG. 43 is an isometric view of the illustrative suture anchors 4002 in delivery sheaths 4004 engaging the tissue 900 with suture control lines 4302 attached in accordance with one aspect of the present disclosure. The suture control lines 4302 may be used for holding sutures in place within the tissue 900 near the guidewire 102 while operations are being performed. The delivery sheaths have been removed to expose the suture control lines 4302.

FIG. 44 is an isometric view of an illustrative cutting implement 4400 in a sheathed position in accordance with one aspect of the present disclosure. The cutting implement 4400 may be tucked away into the sheath 4402. The cutting implement 4400 may be circuitous with a guidewire, that is, its guidance may be based on the guidewire to the tissue.

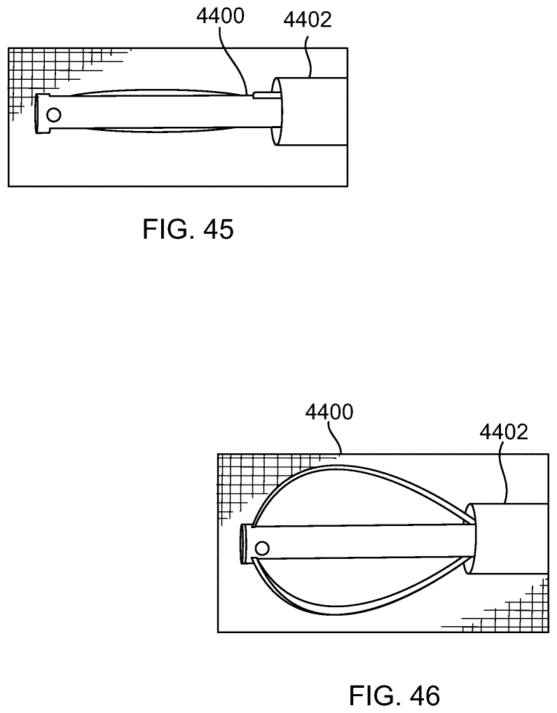

Turning to FIG. 45, an isometric view of the illustrative cutting implement 4400 in an unsheathed position in accordance with one aspect of the present disclosure is provided. The cutting implement 4400 may be extended outside the sheath 4402. This may be performed at a proximal end by an advancing mechanisms through the sheath 4402.

FIG. 46 is an isometric view of the illustrative cutting implement 4400 in an unsheathed position and expanded in accordance with one aspect of the present disclosure. The cutting implement 4400 may be spring loaded such that after extending outside the sheath 4402, it may be expand symmetrically. The cutting implement 4400 may also be expanded through a pull-wire or other mechanism. Expanding the cutting implement 4400 may facilitate tissue incision.

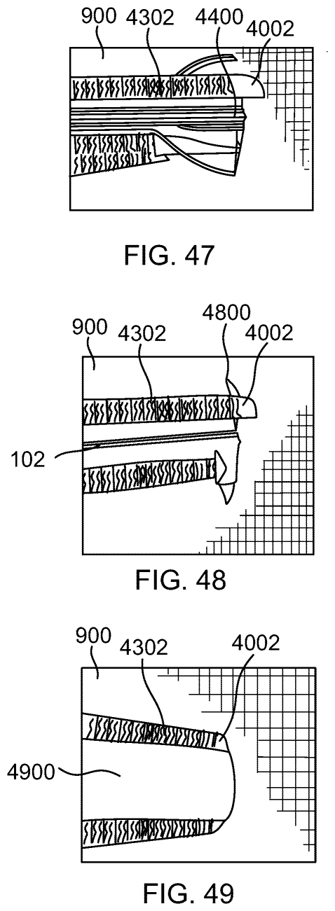

FIG. 47 is an isometric view of the illustrative cutting implement 4400 making an incision or cut into the tissue 900 in accordance with one aspect of the present disclosure. The suture anchors 4002 in the delivery sheaths may be used to hold the tissue 900 in place while allowing guidance of the cutting implement 4400. The cutting implement 4400 may make incisions between the two suture anchors 4002 in the delivery sheaths.

Referring to FIG. 48, an isometric view of an illustrative incision 4800 in the tissue 900 with a guidewire 102 passing through it in accordance with one aspect of the present disclosure is provided. With the cutting implement removed, the two suture anchors 4002 coupled to the suture control lines 4302 along with the guidewire 102 remain.

FIG. 49 is an isometric view of an advancing of an exemplary therapeutic instrument 4900 passing through the incision in the tissue 900 over the guidewire in accordance with one aspect of the present disclosure. The therapeutic instrumentation 4900 may be advanced through the guidewire and through the incision in the tissue 900. The therapeutic instrumentation 4900 may be disposed between the two suture anchors 4002 coupled to the suture control lines 4302.

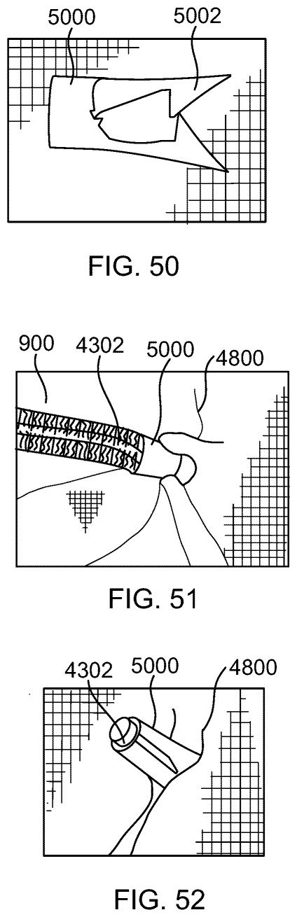

FIG. 50 is an isometric view of an illustrative tissue anchor lock 5000 with helical barbs 5002 in accordance with one aspect of the present disclosure. The lock 5000 with helical barbs 5002 may be implanted into the tissue, which will be shown below, to close an incision.

Turning to FIG. 51, an isometric view of the illustrative tissue anchor lock 5000 with helical barbs engaged in the tissue 900 passed over the suture control lines 4302 in accordance with one aspect of the present disclosure is provided. In this technique, the tissue 900, having the incision 4800, may be twisted with the tissue anchor lock 5000 using the helical barbs. This may secure the sutures, anchors and tissue 900 to one another.

FIG. 52 is an isometric view of the illustrative tissue anchor lock 5000 with helical barbs engaged in the tissue passed over the suture control lines 4302 trimmed to the level of the anchor in accordance with one aspect of the present disclosure. The excess suture control lines 4302 may be trimmed through a separate cutting implement that is guided to the tissue using the guidewire. The incision 4800 may be closed with the lock 5000 which has minimal control lines 4302 attached thereto.

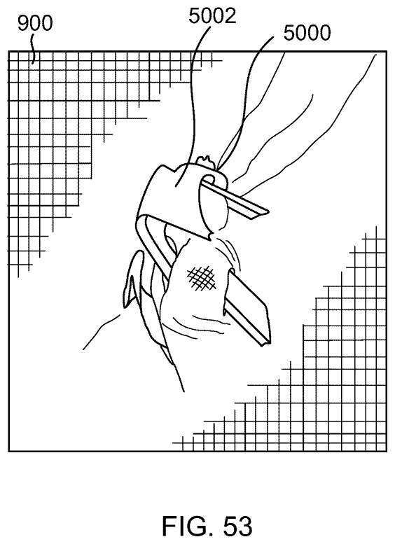

FIG. 53 is an isometric view of the illustrative tissue anchor lock 5000 with helical barbs 5002 engaged in the tissue 900 from the other side of the tissue 900 in accordance with one aspect of the present disclosure. The lock 5000 may be interspersed between the tissue 900 to either heal with the tissue 900 and endothelize, or be absorbed or dissolve. The view is on an opposite side of atrial wall that was shown in FIG. 52. The lock 5000 may grab or hook tissue 900 and then be twisted to seal the incision.

Multiple techniques were described above for closing an incision and allowing re-access. In addition, FIGS. 54-69 provide a fourth embodiment localizing a plurality of pledgets made of biodegradable materials secured together with a knot to close the incision while allowing re-access at a later time. In the following, two pledgets may be used, however, fewer or more may be passed into the tissue for securing the area. The pledgets may be constructed from a bioabsorbable, biodegradable material including but not limited to; sugars, salts, collagen, PLGA, PLLA, other absorbable polymers, magnesium, and or other materials. The pledgets may be constructed from a combination of materials to facilitate different structural properties. Some of these materials may be traditional implant materials such as metals or polymers including, but not limited to, stainless steel, nitinol, cobalt chrome, PEEK, HDPE, and others.

Turning to FIG. 54, an isometric view of an illustrative pledget 5402 made from biocompatible or bio-absorbable material in accordance with one aspect of the present disclosure is provided. The pledget 5402, made from biocompatible or bioabsorbable material, may have a control line 5404 interlaced throughout. The pledget 5402 may be a single piece of material that is elongated with the control lines 5404 dispersed between the pledget 5402 and tied or fastened at the end of the pledget 5402. The control lines 5404 may be interspersed between apertures within the pledget 5402.

FIG. 55 is an isometric view of an exemplary cannula 5500 piecing heart tissue 900 in accordance with one aspect of the present disclosure. The cannula 5500 may have a sharp distal end, but preferably uses RF energy to pierce the tissue 900. The cannula 5500 may use RF at a distal end with insulation covering the tubular structure. The tubular structure may allow a pledget, or other mechanism, to be distributed therethrough. The cannula 5500 may be inserted into a vein, such as the femoral vein, to reach the patient's heart. The cannula 5500 may be set firmly in place. Different variations of cannulas 500 exist and the technique described herein is not limited to the cannula 500 shown.

FIG. 56 is an isometric view of the exemplary cannula 5500 piecing heart tissue 900 and the illustrative pledget 5402 made from biocompatible or bioabsorbable material advanced through the cannula 5500 in accordance with one aspect of the present disclosure. The cannula 5500 may pierce the tissue 900 with a circular cut. Typically, the cut may be small such that the pledget when compacted may plug or fill the cut, providing sufficient apposition to then tension the tissue for later closure. Other shapes for the cut may be used depending on the cross-section of the cannula 5500.

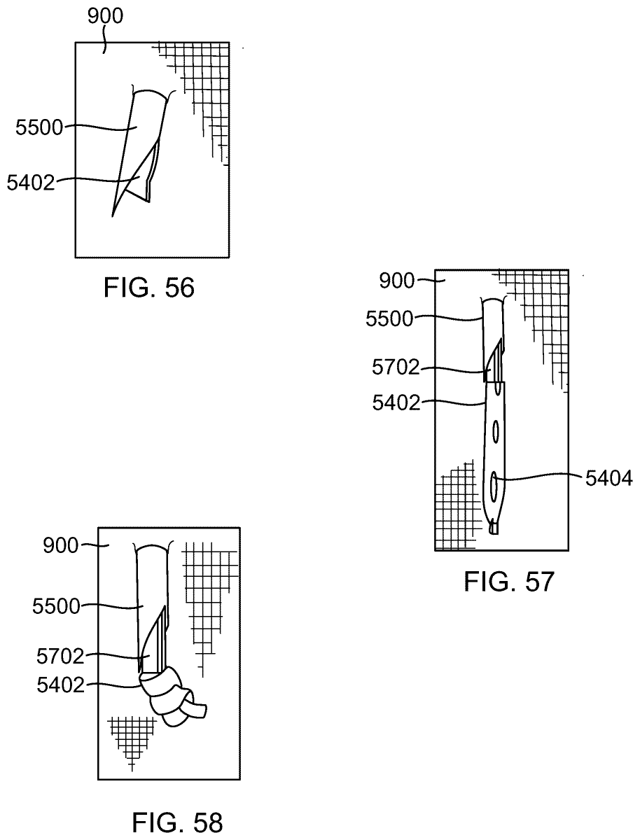

Referring to FIG. 57, an isometric view of the exemplary cannula 5500 piecing heart tissue 900 and the illustrative pledget 5402 made from biocompatible or bioabsorbable material advanced out of the cannula 5500 in accordance with one aspect of the present disclosure is provided. An advancing member 5702 may be placed into the cannula 5500 from a proximal end to force the pledget 5402 with control lines 5404 into the left atrium. In one embodiment, a proximal end of the pledget 5402 is coupled to the distal end of the advancing member 5702 through the control lines 5404 for activation of the pledget by shortening the distance and expanding the cross sectional area of the pledget.

FIG. 58 is an isometric view of the exemplary cannula 5500 piecing heart tissue 900 and the illustrative pledget 5402 made from biocompatible or bioabsorbable material advanced out of the cannula 550 and a pull member tensioned to shorten the pledget 5402 in accordance with one aspect of the present disclosure. The control line 5404 may be pulled through the advancing member 5702. The control line 5404, which was interspersed between apertures within the pledget 5402, may then cause the pledget 5402 to be retracted or shortened. This may cause the pledget 5402 to be bunched up.

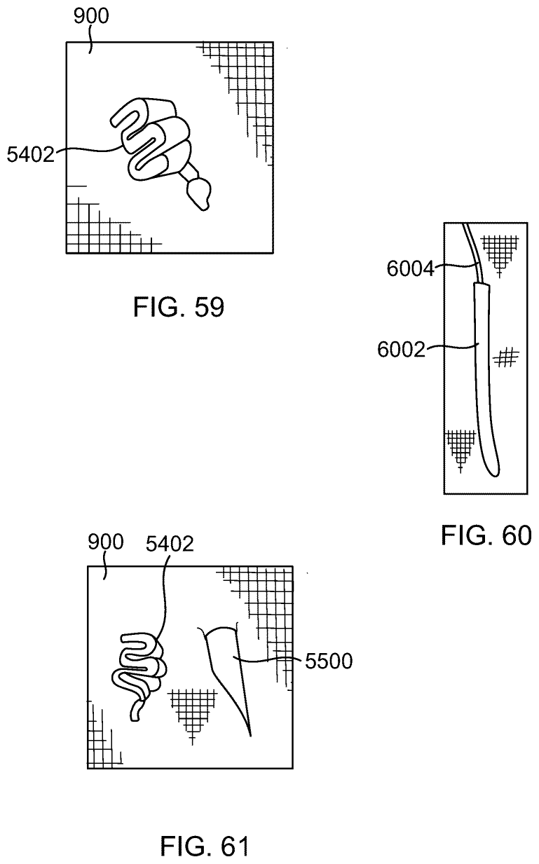

FIG. 59 is an isometric view of the illustrative pledget 5402 made from biocompatible or bioabsorbable material tensioned to shorten the pledget 5402 with the exemplary cannula piecing heart tissue 900 withdrawn and retained on a heart tissue surface in accordance with one aspect of the present disclosure. With the cannula withdrawn and the pledget 5402 shortened and compacted, the initial cut made by the cannula has been plugged or secured.

Referring to FIG. 60, an isometric view of an illustrative concentric pledget 6002 made from biocompatible or bioabsorbable material in accordance with one aspect of the present disclosure is provided. The concentric pledget 6002, made from biocompatible or bioabsorbable material, may be introduced in a similar manner as the other pledget. The concentric pledget 6002 may be made of the same or similar materials as the other pledget. A control line 6004 may be coupled to the concentric pledget 6002 allowing the pledget 6002 to be pulled back such that the concentric pledget 6002 may become contracted or bundled expanding the cross sectional area to provide for greater apposition force to the tissue plane.

FIG. 61 is an isometric view of the exemplary cannula 5500 piecing heart tissue 900 next to the pledget 5402 in accordance with one aspect of the present disclosure. The cannula 5500 may be placed into the tissue 900 near the other pledget 5402. Typically, and as described earlier, the pledgets may be inserted by one another to maintain the integrity of the surrounding tissue as well as allow re-access. The placement may provide for a cutting implement to be disposed between the pledgets to make an incision as well as for a therapeutic or diagnostic instrument to be inserted into the incision.

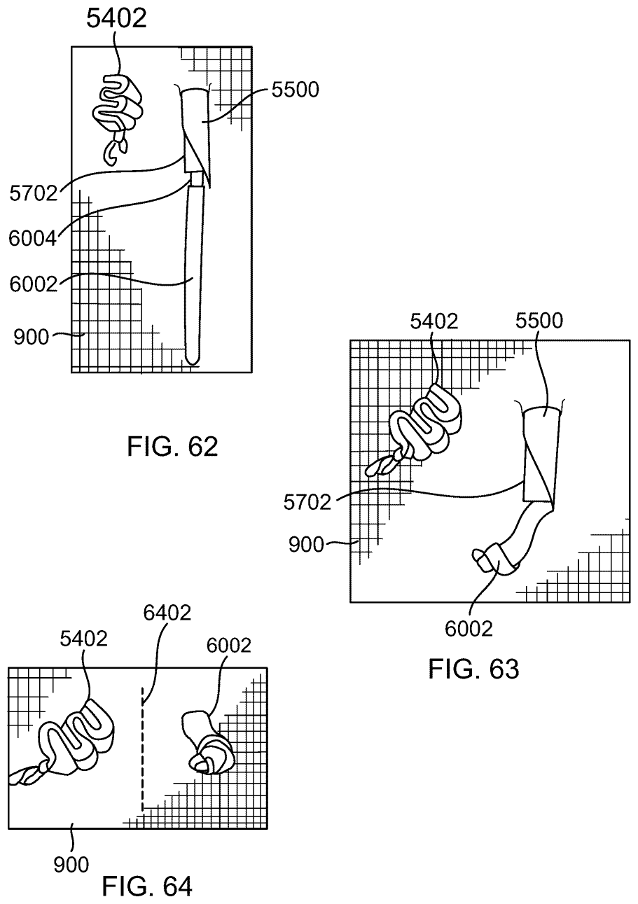

FIG. 62 is an isometric view of the exemplary cannula 5500 piecing heart tissue 900 and the illustrative concentric pledget 6002 made from biocompatible or bioabsorbable material advanced out of the cannula 5500 in accordance with one aspect of the present disclosure. The advancing member 5702 may be used to push or advance the pledget 6002 through the cannula 5500. The pledget 6002 connected to the control line 6004 may be pushed through the tissue 900 via the cannula 5500 next to the other pledget 5402.

FIG. 63 is an isometric view of the exemplary cannula 5500 piecing heart tissue 900 and the illustrative concentric pledget 6002 made from biocompatible or bioabsorbable material advanced out of the cannula 5500 and a pull member tensioned to shorten the pledget in accordance with one aspect of the present disclosure. The control line of the concentric pledget 6002 may be pulled through the advancing member 5702. By doing so, the concentric pledget 6002 may be shorted or bundled.

Referring to FIG. 64, an isometric view of the illustrative concentric pledget 6002 made from biocompatible or bioabsorbable material tensioned to shorten the pledget 6002 and an exemplary incision 6402 made between the pledgets 5402 and 6002 in accordance with one aspect of the present disclosure is provided. The pledgets 5402 and 6002 may provide anchor points where the tissue 900 may be tied together.

A cutting implement, as previously described, may be used to make the incision 6402 between them. While a straight cut is shown, other types of incisions 6402 may be made. These may include, but are not limited to, a straight cut, v cut, zig zag, or crescent arc. With both tissue securing pledgets 5402 and 6002 in place, the incision 6402 may then be made.

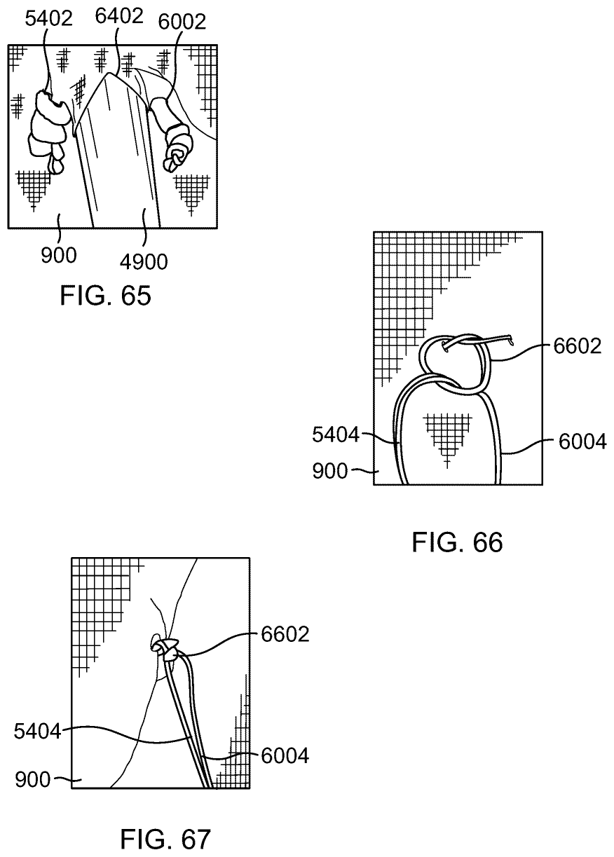

FIG. 65 is an isometric view of the exemplary therapeutic instrument 4900 placed into the incision 6402 between the pledgets 5402 and 6002 into the tissue 900 in accordance with one aspect of the present disclosure. Widening of the incision 6402 may occur when the therapeutic instrument 4900 is placed therein. The therapeutic instrument 4900 may follow a guidewire that was inserted for the other mechanisms brought to the right atrium. A diagnostic instrument may also be used.

FIG. 66 is an isometric view of an illustrative knot 6602 advanced up to the heart tissue 900 with the two pledget control lines 5404 and 6004 in accordance with one aspect of the present disclosure. Upon use and removal of the therapeutic instrument 4900, the securing knot 6602 may be advanced through tightening of the control lines 5404 and 6004 of the pledgets.

Referring to FIG. 67, an isometric view of the illustrative knot 6602 advanced up to the heart tissue 900 with the two pledget control lines 5404 and 6004 and tensioned tight from the knot side of the tissue 900 in accordance with one aspect of the present disclosure is provided. When pulled, the control lines 5404 and 6004 may advance the knot 6002 further to the tissue 900. The control lines 5404 and 6004 may be cut to shorten them, which will be shown below.

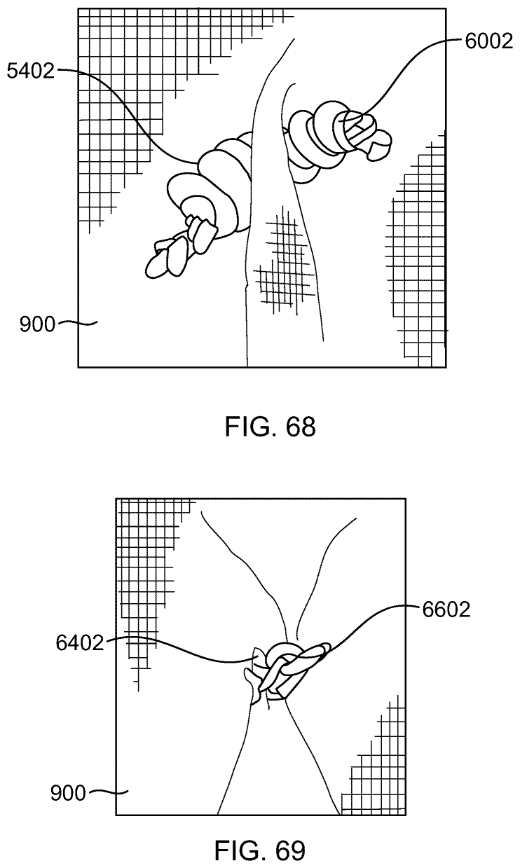

FIG. 68 is an isometric view of the illustrative knot advanced up to the heart tissue 900 with the two pledget tension lines tightened from the pledget side of the tissue 900 in accordance with one aspect of the present disclosure. When tightened, the pledgets 5402 and 6002 may collapse towards one another with tissue 900 folded therebetween.

FIG. 69 is an isometric view of the illustrative incision 6402 closed between the pledgets in accordance with one aspect of the present disclosure. Control lines used to make the knot 6602 may be removed or cut. This may remove any interference that they have within the patient.

In addition to suture and mechanical apparatuses to join the tissue edges together, adhesive materials may be used to seal or join the tissue either as a primary or supplementary or adjunct mechanism. These material may include, but are not limited to, adhesive cyanoacrylates, methoxypropyl cyanoacrylates, alkyl cyanoacrylates such as n-butyl, isobutyl or n-octyl cyanoacrylates, octylcyanoacrylate, butylcyanoacrylate, BioGlue® Surgical Adhesive (BioGlue), bovine serum albumin (BSA), glutaraldehyde of purified (BSA), extracellular matrix ECM human connective tissue, autologous and homologous fibrin sealants, fibrin glue, Polyethylene glycol (PEG)-Based Hydrogel Sealants, hydrogel, methacryloyl-substituted tropoelastin (MeTro), and many others. These sealants may be biocompatible and resorbable.

In one embodiment, a ‘bipolar’ catheter type mechanism may be used to seal the tissue back together. For example, bipolar coagulating forceps used to stop a bleeding vessel may be used. There may be procedural order options to accomplish this procedure.

FIG. 70 is an illustrative flow chart showing exemplary processes for allowing a large bore transseptal access with subsequent atrial re-access in accordance with one aspect of the present disclosure. These processes are for illustrative purposes and may be modified according to those techniques described herein. The processes may begin at block 7000.

At block 7002, a guidewire may be inserted into the heart chamber and distributed at the atrial septum. The guidewire may be inserted into the venous circulatory system through the vascular introduction sheath. The initial percutaneous puncture or incision, by way of example, may be at the patient's femoral vein. Other areas where the guidewire may enter into the patient may include, but is not limited to, a jugular vein, subclavian artery, subclavian vein, or brachial artery and vein.

The guidewire may be routed cranially up the inferior vena cava to the right atrium of the heart. The guidewire may be placed at the atrial septum so that it is used to direct therapeutic or diagnostic catheters into a region of the heart. In turn, the guidewire may be temporarily or removably fixed at the atrial septum.

At block 7004, a puncture may be made by a needle through the atrial septum into the left atrium. The septal penetrator may be a needle or axially elongate structure with a sharp, pointed distal end. In one embodiment, the septal penetrator may be resident within the guidewire. The septal penetrator may be actuated at the proximal end of the vascular device through a control mechanism such as a button, lever, handle, or trigger which may be affixed, permanently or removably by way of a linkage, pusher rod, electrical bus, or the like that runs the length of the device.

The guidewire may be used as the puncture device. The guidewire may have a tip that facilitates the crossing of the septum such as, but not limited to, a sharp end, a helical end, a RF energy electrode tip, other energy tip, or other device to aid the tissue penetration.

Sutures may be pulled through the punctures at block 7006 through an anchor. The sutures may be bundled within a suture bundle and stored in the recess of the anchor. The catheter shaft may be positioned in the right atrium with the anchor, having the suture bundle, advanced into the left atrium through the puncture. In turn, the suture bundle may be unspooled by snares which capture needles coupled to the end of the sutures. The snares with the needles may be pulled into the catheter shaft. The sutures from the suture bundles may be managed through the snares.

Sutures may be placed from the right to left atrium or from the left to right atrium depending on the apparatus. The suture may be placed repeatedly across the septum for multiple points of engagement. The sutures may be another type of apparatus such as a helical anchor or barb device. The placement of the sutures may also be provided after block 7012, when the therapy is complete.

At block 7008, and after the sutures are in place, a cut may be made at the interatrial septum using the cutting implement proximal to the needle passing. The sutures may be deployed through holes made near the initial puncture. Cuts or incisions that are made may be parallel such that the sutures are not cut by the cutting implement. The cutting implement may be coupled to the catheter to make sure that the cutter does not accidentally cut the sutures.

Various cutting implements were described herein. Mechanical or RF energy may be used. When using electrical energy, the amount of exposed metal may be minimized through insulation such that the exposed metal may only exist in the desired tissue cutting region of the tool. Mechanical energy may use blades that may be deployed in the singular direction or may be symmetrical. The cutting may be performed from the right to left atrium or from the left to right depending on the apparatus. The cutting may be post anchor placement. Alternatively, the cutting may precede the suture anchor placement, described at block 7006. The cutting may be integrated into blocks 7002 and 7004 above with a device that punctures and cuts the tissue.

At block 7010, the sutures may be managed. That is, the sutures may be pulled towards the vessel wall and be shifted or manipulated such that they do not interfere or entangle with the therapy instrumentation catheters. The tissue suture management lines may be managed in a lumen of an access sheath or in a separate catheter.

Therapy may be performed using the therapeutic instrument at block 7012. The therapeutic instrument may perform diagnosis and therapeutic intervention to correct atrial fibrillation, perform mitral valve repair, correct septal defects, perform implantation of a cardiac prosthesis, or the like. The therapy or diagnosis may be performed in the left atrium. In turn, the therapeutic instrument may be removed.

At block 7014, the control sutures may be used to close the incision. The amount of this closure may be adjusted to meet the desired therapeutic goals of the procedure. It may be desirable to completely seal the incision, or leave a passage for relief of excess pressure from one side to the other. In one example, the control sutures may be pressed against the tissue which was described above. The processes may end at block 7016.

Other techniques may be used for allowing large bore transseptal access with subsequent atrial re-access. For example, and in this embodiment, the method may include puncturing the septum for needle passing, leaving sutures or some other anchor behind, and then performing a procedure. The anchors or sutures left behind may be cinched together to close the septum. Excess sutures may be then be cut.

In another technique for atrial re-access, the septum may be cut first. The septum may be stabilized through a needle puncture radius so that the needles may pass through the previous cut septum. The needle passing device may be introduced. Needles on the left atrium side may then be snared/grabbed and pulled through the catheter. In turn, pre-shaped nitinol wire may be used to loop through the septum. Control structures may be used to close the incision with the excess sutures cut.

In yet another technique, the transseptal access may be obtained via a standard transseptal approach. The guidewire may be left across the transseptal access site in the left atrium. The vascular device for slicing, enlarging and placing sutures is advanced over the guidewire. Four cutting members radially spaced may slice the septum and enlarge the transseptal access point in a controlled and consistent manner. Needles may then puncture the septum and pass a suture through the interatrial tissue in a location between the slices such that when cinched together they most optimally close the iatrogenic ASD. The vascular device may then be removed, leaving the four sutures in place across the septum. The sutures may be pulled out of the vein and remain temporarily in place in the inferior vena cava vein until later in the procedure.

In yet another technique the mechanism to facilitate the delivery of the suture, anchors, cutting implements, and closing features may be integrated into the therapeutic instrument to minimize the exchange of devices in the patient.

In yet another technique, no foreign body may be left behind. The technique, with associated device or devices, may come into the atrium after the procedure and cinch the tissue to be apposed for a time to promote healing while sealing them. In turn, the technique may include removing the structure or apparatus. This technique may be the combination of a controlled incision and then later cinching with an apparatus described herein, or that is known in the art. The apparatus may include, but is not limited to, grabbers, forceps, helical anchors, pinchers, knots, suction devices, barbs, or the like. This technique may then promote the tissue to heal by itself due to cut morphology. The time of this temporary apposition may range from minutes to days, or weeks, depending on the amount of tissue healing desired.

In some embodiments, the anchors described above may subsequently be removed from the tissue, or alternatively may be left in place. The techniques or procedures for removing the anchors may use grabbers, snares, cutting elements, or engagement features specific to the mechanism left in place. A mechanical feature may be added on the right atrial side of the anchoring device such that it may be subsequently grabbed and unscrewed. This mechanical feature may be in the shape of a hook, an oval, or the like. This feature may protrude off of the right atrial septum such that it may be grasped with a snare and rotated out of the tissue, for example.

The foregoing description is provided to enable any person skilled in the relevant art to practice the various embodiments described herein. Various modifications to these embodiments will be readily apparent to those skilled in the relevant art and generic principles defined herein may be applied to other embodiments. Thus, the claims are not intended to be limited to the embodiments shown and described herein, but are to be accorded the full scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the relevant art are expressly incorporated herein by reference and intended to be encompassed by the claims. Moreover, nothing disclosed herein is intended to be dedicated to the public regardless of whether such disclosure is explicitly recited in the claims.

What is claimed is:

1. A vascular device for performing a transseptal puncture, comprising:
a body;
an anchor extending from a distal end of the body through a shaft disposed within the body;
at least one suture coupled to at least one needle within the anchor;
at least one catch extending from the body to pull the at least one needle into the body for placing the at least one suture; and
a cutting implement between the body and anchor coupled to an actuating shaft aligned with the at least one suture.

2. The vascular device for performing the transseptal puncture of claim 1, comprising a guidewire disposed within the body and anchor.

3. The vascular device for performing the transseptal puncture of claim 1, wherein a face of the body and anchor are angled.

4. The vascular device for performing the transseptal puncture of claim 1, wherein the body comprises a rectangular-shaped lumen for the shaft of the anchor.

5. The vascular device for performing the transseptal puncture of claim 1, wherein the body comprises at least one lumen for the at least one catch.

6. The vascular device for performing the transseptal puncture of claim 1, wherein the at least one suture is stored in a recess channel of the anchor.

7. The vascular device for performing the transseptal puncture of claim 6, wherein the at least one suture comprises two ends coupled to two needles extending towards the body.

8. The vascular device for performing the transseptal puncture of claim 6, wherein the recess channel releases the at least one suture after pulled by the at least one catch.

9. The vascular device for performing the transseptal puncture of claim 1, wherein the device comprises two sutures with each suture coupled to two needles.

10. The vascular device for performing the transseptal puncture of claim 1, wherein the at least one catch is pulled from a proximal end of the body.

11. The vascular device for performing the transseptal puncture of claim 1, wherein the actuating shaft of the cutting implement telescopes over the shaft of the anchor.

12. The vascular device for performing the transseptal puncture of claim 1, wherein the cutting implement is expanded radially from the body through the actuating shaft.

13. The vascular device for performing the transseptal puncture of claim 12, wherein the actuating shaft causes the cutting implement to bow at a greater length than a diameter of the body.

14. The vascular device for performing the transseptal puncture of claim 12, wherein the actuating shaft causes a linkage system to expand the cutting implement.

15. The vascular device for performing the transseptal puncture of claim 1, wherein the cutting implement uses mechanical or electrical energy.

16. The vascular device for performing the transseptal puncture of claim 15, wherein the mechanical or electrical energy comes from at least one of a blade, ceramic, electrocautery technique, radio frequency, plasmajet vaporization, ultra-sonic, high voltage vaporization, controlled dilation, heat and cold.

17. A septal orifice closure apparatus allowing re-access, comprising:
a body on a first side of a septal orifice in a septum of a heart;
an anchor on a second side of the septal orifice extending from a distal end of the body through a shaft disposed within the body;
at least one suture coupled to at least one needle disposed within the anchor;
at least one catch extending from the body to pull the at least one needle into the body for placing the at least one suture; and
a cutting implement between the body and anchor coupled to an actuating shaft aligned with the at least one suture.

18. The septal orifice closure apparatus of claim 17, wherein the cutting implement uses mechanical or electrical energy.

19. The septal orifice closure apparatus of claim 17, comprising a guidewire disposed within the body.

20. The septal orifice closure apparatus of claim 17, wherein the cutting implement comprises a first circuit for a first puncture and a second circuit for a larger cut.
